(19) United States
(12) Patent Application Publication
(10) Pub. No.: US 2026/0315395 A1
(43) Pub. Date: Oct. 15, 2026

(54) SOLID DOSAGE FORMS INCLUDING NON-BLENDED PORCINE THYROID POWDER AND METHODS OF MAKING AND USING THE SAME

(71) Applicant: Genus Lifesciences Inc., Allentown, PA (US)

(72) Inventors: Jeffrey M. Moshal, Allentown, PA (US); Michael Libman, Bradenton, FL (US); Prakash Ramalingam, Fogelsville, PA (US)

(21) Appl. No.: 19/636,339

(22) Filed: Apr. 1, 2026

Related U.S. Application Data
(60) Provisional application No. 63/789,054, filed on Apr. 15, 2025.

(51) Int. Cl. A61K 9/20; A61K 9/00; A61K 31/198; A61K 35/55

(52) U.S. Cl. CPC A61K 9/2054; A61K 9/0053; A61K 9/2009; A61K 9/2018; A61K 9/2095; A61K 31/198; A61K 35/55

(57) ABSTRACT

Oral solid dosage forms including non-blended porcine thyroid powder and methods of making and using the oral solid dosage forms are disclosed.

### FIELD OF THE TECHNOLOGY

The present disclosure relates to pharmaceutical oral solid dosage forms comprising liothyronine salt and levothyroxine salt and to methods of making and using the oral solid dosage forms.

### DESCRIPTION OF THE BACKGROUND OF THE TECHNOLOGY

Hypothyroidism is a common endocrine disorder characterized by a deficiency of thyroid hormone. Worldwide, iodine deficiency is the major cause of hypothyroidism. In the United States and other areas in which iodine intake is adequate, autoimmune thyroid disease (Hashimoto's disease) is the most common cause of hypothyroidism. Hypothyroidism may also be drug-induced or otherwise iatrogenic.

Hypothyroidism usually is a primary process in which the thyroid gland is unable to produce sufficient amounts of thyroid hormone. Hypothyroidism can also be a secondary process in which the thyroid gland is normal but receives insufficient stimulation because of low secretion of thyrotropin (i.e., thyroid-stimulating hormone or “TSH”) by the pituitary gland. Also, in tertiary hypothyroidism, the hypothalamus releases inadequate levels of thyrotropin-releasing hormone (TRH), which leads to insufficient release of TSH, and in turn causes inadequate thyroid stimulation.

Thyroid hormone deficiency has a wide range of effects. Systemic effects result either from derangements in metabolic processes or directly by myxedematous infiltration (i.e., accumulation of glucosaminoglycans in the tissues). Hypothyroid changes in the heart result in decreased contractility, cardiac enlargement, pericardial effusion, decreased pulse, and decreased cardiac output. In the gastrointestinal tract, achlorhydria and prolonged intestinal transit time with gastric stasis can occur. Delayed puberty, anovulation, menstrual irregularities, and infertility are common. Decreased thyroid hormone also can cause increased levels of total cholesterol and low-density lipoprotein (LDL) cholesterol and a possible change in high-density lipoprotein (HDL) cholesterol because of a change in metabolic clearance. In addition, hypothyroidism may result in an increase in insulin resistance.

Third-generation TSH assays are readily available and are generally the most sensitive screening tool for primary hypothyroidism. The generally accepted reference range for normal serum TSH is 0.40-4.2 mIU/L. If TSH levels are above the reference range, the next step is to measure free thyroxine (T4). Subclinical hypothyroidism, also referred to as mild hypothyroidism, is defined as normal serum levels of free T4 and triiodothyronine (T3) with a slightly high serum TSH concentration.

Daily administration of thyroid hormone drugs is the standard treatment for hypothyroidism in humans and other mammals. An oral medication such as levothyroxine or a levothyroxine salt can restore adequate hormone levels, reversing the symptoms of hypothyroidism. In general, hypothyroidism can be adequately treated with a constant daily dose of levothyroxine (LT4). The typical human daily dosage of a levothyroxine drug is low, ranging from 25 to 300 μg. The treatment goals for hypothyroidism are to reverse clinical progression and correct metabolic derangements, as evidenced by normal blood levels of TSH and T4.

Careful dosing of levothyroxine drugs is critical to avoid over and under treatment. Symptoms of overtreatment can include, for example, tachycardia, palpitations, atrial fibrillation, nervousness, tiredness, headache, increased excitability, sleeplessness, tremors, and possible angina. Dosage resulting in under treatment of patients can result in, for example, fatigue, cold intolerance, dry skin, hair loss, muscle pain, joint pain, weakness in the extremities, mental impairment, decreased perspiration, paresthesia and nerve entrapment syndromes, blurred vision, and hearing impairment.

Thyroid hormone replacement is commonly started at anticipated full replacement dosage level in young and otherwise healthy patients. In elderly patients and those with known ischemic heart disease, treatment often begins with one-quarter to one-half anticipated full replacement dosage, and dosage is adjusted upwardly in small increments after no less than 4 to 6 weeks. For most cases of mild to moderate hypothyroidism, a starting levothyroxine dosage is 50-75 μg/day.

All thyroid hormone preparations, natural or synthetic, are intended to replace a patient's natural thyroid hormone. A normal human thyroid gland releases primarily tetraiodothyronine (levothyroxine or LT4), which is then converted to triiodothyronine (liothyronine or T3) in the target tissues. T3 is the actual active thyroid hormone. The thyroid gland forms LT4 (containing four iodine atoms) by coupling two molecules of diiodotyrosine (DIT). T3 (containing three iodine atoms) is also formed in the thyroid gland by coupling one molecule of DIT with one molecule of monoiodotyrosine (MIT). The thyroid gland stores both T4 and T3 in the thyroid colloid as thyroglobulin.

The normal thyroid gland contains approximately 200 μg of levothyroxine (T4) per gram of gland and 15 μg of liothyronine (T3) per gram. The ratio of these two hormones in the circulation does not represent the ratio in the thyroid gland since about 80 percent of peripheral liothyronine (T3) comes from monodeiodination of levothyroxine (T4). Peripheral monodeiodination of levothyroxine (T4) at the 5 positions (inner ring) also results in the formation of reverse liothyronine (T3), which is calorigenically inactive.

Synthetic thyroid hormone preparations are commercially available for both T3 and T4 forms of thyroid hormone. For example, liothyronine sodium is a synthetic version of the T3 thyroid hormone triiodothyronine available as CYTOMEL® tablets from King Pharmaceuticals, St. Louis, MO. Levothyroxine (T4) sodium is available in several commercial products including LEVOXYL® tablets (King Pharmaceuticals), UNITHROID® tablets (Jerome Stevens Pharmaceuticals, Bohemia, NY), and SYNTHROID® tablets (AbbVie, Chicago, IL). Levothyroxine (T4) sodium is also available in a veterinary preparation sold under the trademark SOLOXINE® (King Pharmaceuticals).

Levothyroxine is relatively unstable and degrades over time. For example, levothyroxine sodium is sensitive to irradiation, hydrolysis, oxidation, and heat. Degradation of the active ingredients in thyroxine dosage forms can lead to loss of potency, resulting in inadequate dosage delivery and under treatment of hypothyroidism. Some drug manufacturers have resorted to including an excess of the active ingredient in thyroxine dosage forms in anticipation of some degree of storage degradation. This practice can complicate accurate dosing and present the possibility of over dosing. Accordingly, storage stability of levothyroxine drugs is highly desirable to ensure accurate patient dosing and avoid the complications associated with under or over treatment.

Levothyroxine sodium tablets are available in extremely low, microgram-level dosages (e.g., 25 μg, 50 μg, 75 μg, 88 μg, 100 μg, 112 μg, 125 μg, 137 μg, 150 μg, 175 pg. 200 μg, and 300 μg). To facilitate accurate administration, commercially available tablets typically include a break-line allowing one to break tablets into two substantially equal halves. The possibility that a patient will utilize break-lines and administer only half of a tablet daily enhances the need for uniform distribution of active ingredients in tablets to avoid incorrect dosage and related complications.

Preparations containing natural thyroid hormones also are available. Prior to the availability of pure levothyroxine, desiccated animal thyroid extract was the only available treatment for hypothyroidism. Some individuals prefer desiccated thyroid extract as a more “natural” thyroid hormone. In addition, some patients who continue to have symptoms of hypothyroidism when taking levothyroxine report improvement in these symptoms when switched to desiccated thyroid extract. Thyroid powder is a natural preparation derived from porcine thyroid glands containing thyroid hormones T3 (liothyronine) and T4 (levothyroxine). As is the case with synthetic thyroid hormones, naturally derived liothyronine and levothyroxine can degrade over time, and natural thyroid hormone replacement preparations have a shelf life set to better ensure that individual dosage forms supply the desired amount of thyroid hormone to a patient.

Accordingly, there is a need for a thyroid hormone replacement dosage form including liothyronine and levothyroxine and which exhibits advantageous stability and improved shelf life.

### SUMMARY

It is understood that the inventions disclosed and described in this specification are not limited to the embodiments described in this Summary.

Provided herein is a pharmaceutical oral solid dosage form comprising about 7.5 μg to about 300 μg levothyroxine salt; about 1.75 μg to about 75 μg liothyronine salt; about 74% to about 96%, by weight, microcrystalline cellulose; and at least one excipient selected from a lubricant, a glidant, a diluent, a disintegrant, and a coating.

According to one non-limiting aspect, the present disclosure provides a packaged pharmaceutical oral solid dosage form product comprising a pharmaceutical oral solid dosage form comprising, by weight, about 7.5 μg to about 300 μg levothyroxine salt; about 1.75 μg to about 75 μg liothyronine salt; about 74% to about 96%, by weight, microcrystalline cellulose, and at least one excipient selected from a lubricant, a glidant, a diluent, a disintegrant, and a coating; and a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial levothyroxine salt content in the pharmaceutical oral solid dosage form after the pharmaceutical oral solid dosage form has been stored for 2 months at a temperature of about 38° C. to about 42° C. and a relative humidity of about 70% to about 80%.

According to another non-limiting aspect, the present disclosure provides a packaged pharmaceutical oral solid dosage form product comprising a pharmaceutical oral solid dosage form comprising, by weight, about 7.5 μg to about 300 μg levothyroxine salt; about 1.75 μg to about 75 μg liothyronine salt; about 74% to about 96%, by weight, microcrystalline cellulose, and at least one excipient selected from a lubricant, a glidant, a diluent, a disintegrant, and a coating; a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial liothyronine salt content in the pharmaceutical oral solid dosage form after the pharmaceutical oral solid dosage form has been stored for 2 months at a temperature of about 38° C. to about 42° C. and a relative humidity of about 70% to about 80%.

According to another non-limiting aspect, the present disclosure provides a method of treatment to address hypothyroidism. The method comprises administering a pharmaceutical oral solid dosage form to a subject in need thereof, wherein the pharmaceutical oral solid dosage form comprises, by weight: about 7.5 μg to about 300 μg levothyroxine salt; about 1.75 μg to about 75 μg liothyronine salt; about 74% to about 95%, by weight, microcrystalline cellulose; and at least one excipient selected from a lubricant, a glidant, a diluent, a disintegrant, and a coating.

Provided herein is a pharmaceutical oral solid dosage form comprising: about 7.5 μg to about 300 μg levothyroxine salt; about 1.75 μg to about 75 μg liothyronine salt; about 66% to about 90%, by weight, microcrystalline cellulose; and at least one excipient selected from a lubricant, a glidant, a diluent, an antioxidant, and a coloring agent.

According to another non-limiting aspect, the present disclosure provides a packaged pharmaceutical oral solid dosage form product comprising: a pharmaceutical oral solid dosage form comprising, by weight, about 7.5 μg to about 300 μg levothyroxine salt; about 1.75 μg to about 75 μg liothyronine salt; about 66% to about 90%, by weight, microcrystalline cellulose, and at least one excipient selected from a lubricant, a glidant, a diluent, an antioxidant, and a coloring agent; and a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial levothyroxine salt content in the pharmaceutical oral solid dosage form after the pharmaceutical oral solid dosage form has been stored for 6 months at a temperature of about 25° C. to about 27° C. and a relative humidity of about 55% to about 65%.

According to another non-limiting aspect, the present disclosure provides a packaged pharmaceutical oral solid dosage form product comprising: a pharmaceutical oral solid dosage form comprising, by weight, about 7.5 μg to about 300 μg levothyroxine salt; about 1.75 μg to about 75 μg liothyronine salt; about 66% to about 90%, by weight, microcrystalline cellulose, and at least one excipient selected from a lubricant, a glidant, a diluent, a disintegrant, and a coating; a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial liothyronine salt content in the pharmaceutical oral solid dosage form after the pharmaceutical oral solid dosage form has been stored for 6 months at a temperature of about 25° C. to about 27° C. and a relative humidity of about 55% to about 65%.

According to another non-limiting aspect, the present disclosure provides a packaged pharmaceutical oral solid dosage form product comprising: a pharmaceutical oral solid dosage form comprising, by weight, about 7.5 μg to about 300 μg levothyroxine salt; about 1.75 μg to about 75 μg liothyronine salt; about 66% to about 90%, by weight, microcrystalline cellulose, and at least one excipient selected from a lubricant, a glidant, a diluent, an antioxidant, and a coloring agent; and a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial levothyroxine salt content in the pharmaceutical oral solid dosage form after the pharmaceutical oral solid dosage form has been stored for 6 months at a temperature of about 38° C. to about 42° C. and a relative humidity of about 70% to about 80%.

According to another non-limiting aspect, the present disclosure provides a a packaged pharmaceutical oral solid dosage form product comprising: a pharmaceutical oral solid dosage form comprising, by weight, about 7.5 μg to about 300 μg levothyroxine salt; about 1.75 μg to about 75 μg liothyronine salt; about 66% to about 90%, by weight, microcrystalline cellulose, and at least one excipient selected from a lubricant, a glidant, a diluent, a disintegrant, and a coating; a container closure system comprising a container and a sealing closure; provided that a plurality of the pharmaceutical oral solid dosage forms and at least about 2 grams of desiccant and/or at least 2 grams of oxygen absorber are enclosed in the container and sealed with the sealing closure; and provided that each pharmaceutical oral solid dosage form enclosed in the container retains at least 90%, at least 92%, or at least 95% of an initial liothyronine salt content in the pharmaceutical oral solid dosage form after the pharmaceutical oral solid dosage form has been stored for 6 months at a temperature of about 38° C. to about 42° C. and a relative humidity of about 70% to about 80%.

According to another non-limiting aspect, the present disclosure provides a method of treatment to address hypothyroidism. The method comprises administering a pharmaceutical oral solid dosage form to a subject in need thereof, wherein the pharmaceutical oral solid dosage form comprises, by weight: about 7.5 μg to about 300 μg levothyroxine salt; about 1.75 μg to about 75 μg liothyronine salt; about 66% to about 90%, by weight, microcrystalline cellulose; and at least one excipient selected from a lubricant, a glidant, a diluent, an antioxidant, and a coloring agent.

### BRIEF DESCRIPTION OF THE DRAWINGS

Various features and characteristics of non-limiting and non-exhaustive embodiments disclosed and described in this specification may be better understood by reference to the accompanying figures, in which:

FIGS. 1A and 1B, respectively, are side and section views of an embodiment of a 30 cm³ (1.0 oz.) white high-density polyethylene (“HDPE”) bottle used to contain oral solid dosage forms, as described herein.

FIG. 1C is a side view of Detail “A” in FIG. 1A.

FIGS. 2A-2C are views of an embodiment of a container closure that can be used with the container shown in FIGS. 1A, 1B, and 1C, wherein FIG. 2A is an exterior elevational view of a ribbed, threaded closure cap, FIG. 2B is an exterior top view of the ribbed, threaded closure cap, and FIG. 2C is a side sectional view of the cap shown in FIG. 2B.

FIGS. 3A and 3B, respectively, are side and section views of an embodiment of a 75 cm³ (2.5 oz.) white HDPE bottle used to contain oral solid dosage forms, as described herein.

FIG. 3C is a side view of Detail “A” in FIG. 3A.

FIGS. 4A and 4B are views of an embodiment of a container closure that can be used with the container shown in FIGS. 3A, 3B, and 3C, wherein FIG. 4A is an exterior top view of a ribbed, threaded closure cap and FIG. 4B is a side sectional view of the cap shown in FIG. 4A.

The reader will appreciate the foregoing details, as well as others, upon considering the following detailed description of various non-limiting and non-exhaustive embodiments according to the present disclosure.

### DESCRIPTION OF CERTAIN NON-LIMITING EMBODIMENTS

Various embodiments are described and illustrated in this specification to provide an overall understanding of the disclosed compositions and methods. It is understood that the various embodiments described and illustrated in this specification are non-limiting and non-exhaustive. Thus, the present invention is not limited by the description of the various non-limiting and non-exhaustive embodiments disclosed in this specification. Rather, the invention is defined solely by the claims. Certain features and characteristics illustrated and/or described in connection with various embodiments may be combined with the features and characteristics of other embodiments. Such modifications and variations are intended to be included within the scope of this specification. As such, the claims may be amended to recite any features or characteristics expressly or inherently described in, or otherwise expressly or inherently supported by, this specification. Further, Applicant reserves the right to amend the claims to affirmatively disclaim features or characteristics that may be found present in the prior art. The various embodiments disclosed and described in this specification can comprise, consist of, or consist essentially of the features and characteristics as variously described herein.

All percentages provided herein are weight percentages based on the total weight of the respective composition, tablet, mixture, etc., unless otherwise indicated.

Any numerical ranges recited in this specification are intended to include all sub-ranges of the same numerical precision subsumed within the recited range. For example, a range of “1.0 to 10.0” is intended to include all sub-ranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited in this specification is intended to include all lower numerical limitations subsumed therein and any minimum numerical limitation recited in this specification is intended to include all higher numerical limitations subsumed therein. Applicant reserves the right to amend this specification, including the claims, to expressly recite any sub-range subsumed within the ranges expressly recited herein.

As generally used herein, the term “about” refers to an acceptable degree of error for the quantity measured, given the nature or precision of the measurement. Typical exemplary degrees of error may be within 20%, 10%, or 5% of a given value or range of values.

The terms “subject” and “patient” are used interchangeably herein, and it is intended that both refer to a recipient on whom a method is conducted according to the present disclosure or another method, as the case may be.

The term “ready to use” as used herein refers to a composition that is suitable for administration to a patient without further preparation.

The terms “stability” and “stable” as used herein refer to each oral solid dosage form retaining at least 90%, at least 95%, at least 99%, or at least 100% of an initial levothyroxine salt content and/or an initial liothyronine salt content in the pharmaceutical oral solid dosage form after the pharmaceutical oral solid dosage form has been stored for a period of time under indicated conditions.

Disclosed herein are storage stable, pharmaceutical oral solid dosage forms. In certain of the embodiments herein, the pharmaceutical oral solid dosage forms (e.g., tablets) comprise liothyronine salt, levothyroxine salt, microcrystalline cellulose, at least one excipient selected from a lubricant, a glidant, a diluent, and a disintegrant, and a coating. The present inventors observed that certain embodiments of the pharmaceutical oral solid dosage forms according to the present disclosure exhibit unexpectedly advantageous stability of the levothyroxine and liothyronine salts in the drug forms.

The present pharmaceutical oral solid dosage forms can comprise two active pharmaceutical ingredients: salts of levothyroxine (also known as thyroxine and L-thyroxine) and liothyronine (also known as(S)-trilodothyronine), which are both illustrated below.

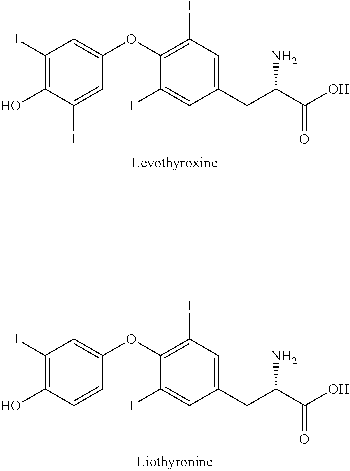 [SMILES: N[C@@H](Cc1cc(I)c(Oc2cc(I)c(O)c(I)c2)c(I)c1)C(=O)O ; N[C@@H](Cc1cc(I)c(Oc2ccc(O)c(I)c2)c(I)c1)C(=O)O]

In certain non-limiting embodiments, depending on the desired strength of the particular pharmaceutical oral solid dosage form, the mass of liothyronine salt included in a pharmaceutical oral solid dosage form according to the present disclosure can range from about 1.75 μg to about 75 μg, such as, for example, from about 1.75 μg to about 2.75 μg, from about 2.45 μg to about 3.75 μg, from about 3.5 μg to about 5.5 μg, from about 5 μg to about 7.5 μg, from about 7 μg to about 11 μg, from about 10 μg to about 15 μg, from about 10 μg to about 16.5 μg, from about 14 μg to about 22 μg, from about 20 μg to about 30 μg, from about 30 μg to about 45 μg, from about 35 μg to about 55 μg, from about 50 μg to about 75 μg, or from about 2 μg to about 69 μg.

In certain non-limiting embodiments, liothyronine salt may be present in the pharmaceutical oral solid dosage forms according to the present disclosure in a concentration of 0.0015% to 0.055%, such as, for example, 0.002% to 0.055%, 0.0025% to 0.055%, 0.005% to 0.055%, 0.0075% to 0.055%, 0.01% to 0.055%, 0.015% to 0.055%, 0.02% to 0.055%, 0.025% to 0.055%, 0.03% to 0.055%, 0.035% to 0.055%, 0.04% to 0.055%, 0.045% to 0.055%, 0.050% to 0.055%, 0.01% to 0.045%, 0.01% to 0.035%, 0.01% to 0.025%, 0.02% to 0.045%, 0.02% to 0.035%, 0.02% to 0.025%, 0.025% to 0.035%, 0.0015% to 0.01%, 0.0015% to 0.0075%, 0.0015% to 0.005%, 0.0015% to 0.003%, 0.002% to 0.01%, 0.002% to 0.0075%, 0.002% to 0.005%, 0.002% to 0.003%, all in weight percent based on total weight of the pharmaceutical oral solid dosage form. In other embodiments, the liothyronine salt may be present in the pharmaceutical oral solid dosage form in a concentration of about 0.0015%, about 0.002%, about 0.0022%, about 0.0023%, about 0.0024%, about 0.0025%, about 0.003%, about 0.004%, about 0.005%, about 0.006%, about 0.007%, about 0.008%, about 0.009%, about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, about 0.03%, about 0.031%, about 0.032%, about 0.033%, about 0.034%, about 0.035%, about 0.036%, about 0.037%, about 0.038%, about 0.039%, about 0.04%, about 0.041%, about 0.042%, about 0.043%, about 0.044%, about 0.045%, about 0.046%, about 0.047%, about 0.048%, about 0.049%, about 0.05%, about 0.051%, about 0.052%, about 0.053%, about 0.054%, or about 0.055%, all in weight percent based on the total weight of the pharmaceutical oral solid dosage form.

In certain non-limiting embodiments, depending on the desired strength of the particular pharmaceutical oral solid dosage form, the mass of levothyroxine salt included in a pharmaceutical oral solid dosage form according to the present disclosure can range from about 7.5 μg to about 300 μg, such as, for example, from about 7.5 μg to about 11.5 μg, from about 10 μg to about 15 μg, from about 15 μg to about 23 μg, from about 20 μg to about 30 μg, from about 30 μg to about 46 μg, from about 40 μg to about 60 μg, from about 45 μg to about 69 μg, from about 60 μg to about 92 μg, from about 80 μg to about 120 μg, from about 120 μg to about 180 μg, from about 150 μg to about 230 μg, from about 200 μg to about 300 μg, or from about 8 μg to about 275 μg.

In certain non-limiting embodiments, levothyroxine salt may be present in the pharmaceutical oral solid dosage form according to the present disclosure in a concentration of 0.005% to 0.25%, such as, for example, 0.01% to 0.25%, 0.015% to 0.25%, 0.02% to 0.25%, 0.025% to 0.25%, 0.03% to 0.25%, 0.035% to 0.25%, 0.04% to 0.25%, 0.045% to 0.25%, 0.05% to 0.25%, 0.055% to 0.25%, 0.06% to 0.25%, 0.065% to 0.25%, 0.07% to 0.25%, 0.075% to 0.25%, 0.08% to 0.25%, 0.085% to 0.25%, 0.09% to 0.25%, 0.1% to 0.25%, 0.11% to 0.25%, 0.12% to 0.25%, 0.13% to 0.25%, 0.14% to 0.25%, 0.15% to 0.25%, 0.16% to 0.25%, 0.17% to 0.25%, 0.18% to 0.25%, 0.19% to 0.25%, 0.20% to 0.25%, 0.21% to 0.25%, 0.225% to 0.25%, 0.005% to 0.1%, 0.006% to 0.1%, 0.007% to 0.1%, 0.008% to 0.1%, 0.009% to 0.1%, 0.01% to 0.1%, 0.025% to 0.1%, 0.05% to 0.1%, 0.075% to 0.1%, 0.075% to 0.125%, 0.125% to 0.2%, 0.15% to 0.2%, or 0.175% to 0.2%, all in weight percent based on the total weight of the pharmaceutical oral solid dosage form.

In certain non-limiting embodiments according to the present disclosure, the weight ratio of levothyroxine salt to liothyronine salt in the oral solid dosage form is about 3.5:1 to about 4.5:1, such as for example, about 3.75:1 to about 4.5:1, about 4.0:1 to about 4.5:1, or about 4.25:1 to about 4.5:1.

Various non-limiting embodiments of a pharmaceutical oral solid dosage form according to the present disclosure can comprise one or more diluents. Diluents may be included as fillers in the pharmaceutical oral solid dosage form (e.g., tablet) to increase dosage form weight and improve content uniformity. Non-limiting examples of suitable diluents that can be used in the pharmaceutical oral solid dosage forms disclosed herein include at least one of microcrystalline cellulose (MCC) (e.g., AVICEL® PH-102 MCC powder), AVICEL® HFE-102 powder (a blend of microcrystalline cellulose and mannitol, available from FMC BioPolymer, Philadelphia, PA), confectioner's sugar (including corn starch), croscarmellose sodium, dicalcium phosphate, inulin, carbohydrates (for example, arabinose, sucrose, dextrose, fructose, maltose, lactose, lactose monohydrate, trehalose, isomalt, starch, monosaccharides, disaccharides, polysaccharides, and sugar alcohols (e.g., sorbitol, mannitol, erythritol, xylitol, lactitol)), derivatives of the foregoing, and combinations of any thereof.

In certain non-limiting embodiments, diluent may be present in pharmaceutical oral solid dosage forms according to the present disclosure in a concentration of about 74% to about 96%, such as, for example, 74% to 93%, 74% to 90%, 74% to 87%, 74% to 84%, 74% to 81%, 74% to 78%, 78% to 93%, 78% to 90%, 78% to 87%, 78% to 86%, 78% to 84%, 80% to 93, 80% to 90%, 80% to 87%, 80% to 84%, 81% to 93%, 81% to 90%, 81% to 87%, 81% to 84%, 82% to 93%, 82% to 90%, 82% to 87%, 82% to 84%, 83% to 93%, 83% to 90%, or 83% to 87%, all in weight percent based on total weight of the pharmaceutical oral solid dosage form. In other embodiments, diluent may be present in the pharmaceutical oral solid dosage forms according to the present disclosure in a concentration of about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, or about 96%, all in weight percent based on total weight of the pharmaceutical oral solid dosage form.

In certain non-limiting embodiments, diluent may be present in pharmaceutical oral solid dosage forms according to the present disclosure in a concentration of about 66% to about 90%, such as, for example, 66% to 87%, 66% to 84%, 70% to 90%, 70% to 87%, 70% to 86%, 70% to 84%, 70% to 81%, 74% to 87%, 74% to 84%, 74% to 82%, 76% to 90%, 76% to 87%, 76% to 84%, or 76% to 82%, all in weight percent based on total weight of the pharmaceutical oral solid dosage form. In other embodiments, diluent may be present in the pharmaceutical oral solid dosage forms according to the present disclosure in a concentration of about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, all in weight percent based on total weight of the pharmaceutical oral solid dosage form.

In certain non-limiting embodiments according to the present disclosure, the amount of diluent included in a pharmaceutical oral solid dosage form can range from about 85 mg to about 130 mg, such as from about 88 mg to about 130 mg, from about 92 mg to about 130 mg, from about 95 mg to about 130 mg, from about 100 mg to about 130 mg, from about 105 mg to about 130 mg, from about 95 mg to about 115 mg, from about 97.5 mg to about 115 mg, from about 100 mg to about 115 mg, from about 102.5 mg to about 115 mg, from about 105 mg to about 115 mg, from about 105 mg to about 112.5 mg, or from about 105 mg to about 110 mg.

In certain non-limiting embodiments, microcrystalline cellulose (MCC) may be present in pharmaceutical oral solid dosage forms according to the present disclosure in a concentration of about 74% to about 96%, such as, for example, 74% to 93%, 74% to 90%, 74% to 87%, 74% to 84%, 74% to 81%, 74% to 78%, 78% to 93%, 78% to 90%, 78% to 87%, 78% to 86%, 78% to 84%, 80% to 93, 80% to 90%, 80% to 87%, 80% to 84%, 81% to 93%, 81% to 90%, 81% to 87%, 81% to 84%, 82% to 93%, 82% to 90%, 82% to 87%, 82% to 84%, 83% to 93%, 83% to 90%, or 83% to 87%, all in weight percent based on total weight of the pharmaceutical oral solid dosage form. In other embodiments, MCC may be present in the pharmaceutical oral solid dosage forms according to the present disclosure in a concentration of about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, or about 96%, all in weight percent based on total weight of the pharmaceutical oral solid dosage form.

In certain non-limiting embodiments, microcrystalline cellulose (MCC) may be present in pharmaceutical oral solid dosage forms according to the present disclosure in a concentration of about 66% to about 90%, such as, for example, 66% to 87%, 66% to 84%, 70% to 90%, 70% to 87%, 70% to 86%, 70% to 84%, 70% to 81%, 74% to 87%, 74% to 84%, 74% to 82%, 76% to 90%, 76% to 87%, 76% to 84%, or 76% to 82% all in weight percent based on total weight of the pharmaceutical oral solid dosage form. In other embodiments, MCC may be present in the pharmaceutical oral solid dosage forms according to the present disclosure in a concentration of about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89% or about 90%, all in weight percent based on total weight of the pharmaceutical oral solid dosage form.

In certain non-limiting embodiments, the amount of MCC included in a pharmaceutical oral solid dosage form can range from about 85 mg to about 130 mg, such as from about 88 mg to about 130 mg, from about 92 mg to about 130 mg, from about 95 mg to about 130 mg, from about 100 mg to about 130 mg, from about 105 mg to about 130 mg, from about 95 mg to about 115 mg, from about 97.5 mg to about 115 mg, from about 100 mg to about 115 mg, from about 102.5 mg to about 115 mg, from about 105 mg to about 115 mg, from about 105 mg to about 112.5 mg, or from about 105 mg to about 110 mg.

AVICEL® HFE-102 powder is a pharmaceutical diluent that is a spray dried blend of about 90 weight percent microcrystalline cellulose and about 10 weight percent mannitol. In certain non-limiting embodiments, AVICEL® HFE-102 powder may be present in pharmaceutical oral solid dosage forms according to the present disclosure in a concentration of about 74% to about 96%, such as, for example, 74% to 93%, 74% to 90%, 74% to 87%, 74% to 84%, 74% to 81%, 74% to 78%, 78% to 93%, 78% to 90%, 78% to 87%, 78% to 86%, 78% to 84%, 80% to 93, 80% to 90%, 80% to 87%, 80% to 84%, 81% to 93%, 81% to 90%, 81% to 87%, 81% to 84%, 82% to 93%, 82% to 90%, 82% to 87%, 82% to 84%, 83% to 93%, 83% to 90%, or 83% to 87%, all in weight percent based on total weight of the pharmaceutical oral solid dosage form. In other embodiments, AVICEL® HFE-102 powder may be present in the pharmaceutical oral solid dosage forms according to the present disclosure in a concentration of about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, or about 96%, all in weight percent based on total weight of the pharmaceutical oral solid dosage form.

In certain non-limiting embodiments, the amount of AVICEL® HFE-102 powder included in a pharmaceutical oral solid dosage form can range from about 85 mg to about 130 mg, such as from about 88 mg to about 130 mg, from about 92 mg to about 130 mg, from about 95 mg to about 130 mg, from about 100 mg to about 130 mg, from about 105 mg to about 130 mg, from about 95 mg to about 115 mg, from about 97.5 mg to about 115 mg, from about 100 mg to about 115 mg, from about 102.5 mg to about 115 mg, from about 105 mg to about 115 mg, from about 105 mg to about 112.5 mg, or from about 105 mg to about 110 mg.

AVICEL® PH-102 powder is a powdered form of MCC commonly used as a pharmaceutical diluent. In certain non-limiting embodiments, AVICEL® PH-102 powder may be present in pharmaceutical oral solid dosage forms according to the present disclosure in a concentration of about 66% to about 90%, such as, for example, 66% to 87%, 66% to 84%, 70% to 90%, 70% to 87%, 70% to 86%, 70% to 84%, 70% to 81%, 74% to 87%, 74% to 84%, 74% to 82%, 76% to 90%, 76% to 87%, 76% to 84%, or 76% to 82%, all in weight percent based on total weight of the pharmaceutical oral solid dosage form. In other embodiments, AVICEL® PH-102 powder may be present in the pharmaceutical oral solid dosage forms according to the present disclosure in a concentration of about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, or about 90%, all in weight percent based on total weight of the pharmaceutical oral solid dosage form.

In certain non-limiting embodiments, the amount of AVICEL® PH-102 powder included in a pharmaceutical oral solid dosage form according to the present disclosure can range from about 85 mg to about 130 mg, such as from about 88 mg to about 130 mg, from about 92 mg to about 130 mg, from about 95 mg to about 130 mg, from about 100 mg to about 130 mg, from about 105 mg to about 130 mg, from about 95 mg to about 115 mg, from about 97.5 mg to about 115 mg, from about 100 mg to about 115 mg, from about 102.5 mg to about 115 mg, from about 105 mg to about 115 mg, from about 105 mg to about 112.5 mg, or from about 105 mg to about 110 mg.

In certain non-limiting embodiments, mannitol may be present in pharmaceutical oral solid dosage forms according to the present disclosure in a concentration of about 9% to about 14%, such as, for example, 9.25% to 14%, 9.5% to 14%, 9.75% to 14%, 10% to 14%, 10.25% to 14%, 10.5% to 14%, 11% to 14%, 9.25% to 13.75%, 9.25% to 13.5%, 9.25% to 13%, 9.25% to 12.5%, 9.25% to 12%, 9.25% to 11.75%, or 9.25% to 11.5%, all in weight percent based on total weight of the pharmaceutical oral solid dosage form. In other embodiments, mannitol may be present in the pharmaceutical oral solid dosage forms according to the present disclosure in a concentration of about 9%, about 9.25%, about 9.5%, about 9.75%, about 10%, about 10.25%, about 10.5%, about 10.75%, about 11%, about 11.25%, about 11.5%, about 11.75%, about 12%, about 12.25%, about 12.5%, about 13%, about 13.25%, about 13.5%, about 13.75%, or about 14%, all in weight percent based on total weight of the pharmaceutical oral solid dosage form.

In certain non-limiting embodiments according to the present disclosure, the amount of mannitol included in a pharmaceutical oral solid dosage form can range from about 5 mg to about 20 mg, such as from about 7.5 mg to about 20 mg, from about 10 mg to about 20 mg, from about 12.5 mg to about 20 mg, from about 14 mg to about 20 mg, from about 7.5 mg to about 18 mg, from about 7.5 mg to about 16 mg, from about 7.5 mg to about 15 mg, from about 10 mg to about 16 mg, from about 12 mg to about 16 mg, from about 14 mg to about 16 mg, or from about 13 mg to about 15 mg.

In certain non-limiting embodiments, mannitol may be present in pharmaceutical oral solid dosage forms according to the present disclosure in a concentration of about 16% to about 24%, such as, for example, 16.5% to 24%, 17% to 24%, 17.5% to 24%, 18% to 24%, 18.5% to 24%, 19% to 24%, 16% to 23.5%, 16% to 23%, 16% to 22.5%, 16% to 22%, 16% to 21.5%, 16% to 21%, 17% to 23%, 17.5% to 22.5%, 18% to 22%, 19% to 22%, or 18% to 21%, all in weight percent based on total weight of the pharmaceutical oral solid dosage form. In other embodiments, mannitol may be present in the pharmaceutical oral solid dosage forms according to the present disclosure in a concentration of about 16%, about 16.5%, about 17%, about 17.5%, about 18%, about 18.5%, about 19%, about 19.5%, about 20%, about 20.5%, about 21%, about 21.5%, about 22%, about 22.5%, about 23%, about 23.5%, or about 24%, all in weight percent based on total weight of the pharmaceutical oral solid dosage form.

In certain non-limiting embodiments according to the present disclosure, the amount of mannitol included in a pharmaceutical oral solid dosage form can range from about 15 mg to about 30 mg, such as from about 17.5 mg to about 30 mg, from about 20 mg to about 30 mg, from about 22.5 mg to about 30 mg, from about 24 mg to about 30 mg, from about 15 mg to about 28 mg, from about 18 mg to about 28 mg, from about 20 mg to about 28 mg, from about 22 mg to about 28 mg, or from about 24 mg to about 28 mg.

In certain non-limiting embodiments, hydroxypropyl methylcellulose may be present in pharmaceutical oral solid dosage forms according to the present disclosure in a concentration of about 2% to about 7%, such as, for example, 2.25% to 7%, 2.5% to 7%, 2.75% to 7%, 3% to 7%, 3.25% to 7%, 3.5% to 7%, 3.75% to 7%, 4% to 7%, 4.25% to 7%, 4.5% to 7%, 2% to 6.75%, 2% to 6.5%, 2% to 6%, 2% to 5.5%, 2% to 5%, or 2% to 4.75%, all in weight percent based on total weight of the pharmaceutical oral solid dosage form. In other embodiments, hydroxypropyl methylcellulose may be present in the pharmaceutical oral solid dosage forms according to the present disclosure in a concentration of about 2%, about 2.25%, about 2.5%, about 2.75%, about 3%, about 3.25%, about 3.5%, about 3.75%, about 4%, about 4.25%, about 4.5%, about 4.75%, about 5%, about 5.25%, about 5.5%, about 5.75%, about 6%, about 6.25%, about 6.5%, about 6.75%, or about 7%, all in weight percent based on total weight of the pharmaceutical oral solid dosage form.

In certain non-limiting embodiments according to the present disclosure, the amount of hydroxypropyl methylcellulose included in a pharmaceutical oral solid dosage form can range from about 1 mg to about 10 mg, such as from about 2 mg to about 10 mg, from about 3 mg to about 10 mg, from about 4 mg to about 10 mg, from about 5 mg to about 10 mg, from about 2 mg to about 9 mg, from about 2 mg to about 8 mg, from about 2 mg to about 7 mg, from about 3 mg to about 7 mg, from about 4 mg to about 7 mg, from about 2 mg to about 5 mg, or from about 2 mg to about 4 mg.

In certain non-limiting embodiments according to the present disclosure, butylhydroxyanisole may be present in pharmaceutical oral solid dosage forms according to the present disclosure in a concentration of about 0.001% to about 0.009%, such as, for example, 0.0015% to 0.009%, 0.002% to 0.009%, 0.0025% to 0.009%, 0.003% to 0.009%, 0.0035% to 0.009%, 0.0015% to 0.0085%, 0.0015% to 0.008%, 0.0015% to 0.0075%, 0.0015% to 0.007%, 0.0015% to 0.0065%, 0.0015% to 0.006%, 0.0015% to 0.0055%, 0.0015% to 0.005%, 0.0015% to 0.0045%, or 0.0015% to 0.004%, all in weight percent based on total weight of the pharmaceutical oral solid dosage form. In other embodiments, butylhydroxyanisole may be present in the pharmaceutical oral solid dosage forms according to the present disclosure in a concentration of about 0.001%, about 0.0015%, about 0.002%, about 0.0025%, about 0.003%, about 0.0035%, about 0.004%, about 0.0045%, about 0.005%, about 0.0055%, about 0.006%, about 0.0065%, about 0.007%, about 0.0075%, about 0.008%, about 0.0085%, or about 0.009%, all in weight percent based on total weight of the pharmaceutical oral solid dosage form.

In certain non-limiting embodiments according to the present disclosure, the amount of butylhydroxyanisole included in a pharmaceutical oral solid dosage form can range from about 0.001 mg to about 0.01 mg, such as from about 0.0015 mg to about 0.01 mg, about 0.002 mg to about 0.01 mg, about 0.0025 mg to about 0.01 mg, about 0.003 mg to about 0.01 mg, about 0.0035 mg to about 0.01 mg, about 0.004 mg to about 0.01 mg, about 0.0045 mg to about 0.01 mg, about 0.001 mg to about 0.0095 mg, about 0.001 mg to about 0.009 mg, about 0.001 mg to about 0.0085 mg, about 0.001 mg to about 0.008 mg, about 0.001 mg to about 0.0075 mg, about 0.001 mg to about 0.007 mg, about 0.001 mg to about 0.0065 mg, about 0.001 mg to about 0.006 mg, about 0.001 mg to about 0.0055 mg, about 0.003 mg to about 0.008 mg, about 0.003 mg to about 0.006 mg, or about 0.004 mg to about 0.006 mg

Various non-limiting embodiments of a pharmaceutical oral solid dosage form according to the present disclosure comprise one or more lubricants. Lubricants may be included in the pharmaceutical oral solid dosage forms, for example, to improve powder processing properties of the formulation when manufacturing the pharmaceutical solid dosage forms. Non-limiting examples of suitable lubricants that can be used in the pharmaceutical oral solid dosage forms according to the present disclosure include sodium stearyl fumarate, polyethylene glycols, mineral oil, medium chain triglycerides, sodium stearyl sulfate, cocoa butter, sodium benzoate, stearic acid (and its derivatives or esters such as, for example, sodium stearate, magnesium stearate, calcium stearate), and combinations of any thereof.

The concentration of lubricant in a pharmaceutical oral solid dosage form in certain non-limiting embodiments according to the present disclosure can range from about 0.6% to about 1%, from about 0.65% to about 1%, from about 0.6% to about 0.95%, from about 0.7% to about 1%, from about 0.7% to about 0.95%, from about 0.75% to about 1%, from about 0.75% to about 0.95%, from about 0.75% to about 0.90%, from about 0.7% to about 0.95%, from about 0.7% to about 0.85%, or from about 0.75% to about 0.85%, all in weight percent based on total weight of the oral solid dosage form.

In certain non-limiting embodiments, the amount of lubricant included in a pharmaceutical oral solid dosage form according to the present disclosure can range from about 0.20 mg to about 0.32 mg, from about 0.21 mg to about 0.32 mg, from about 0.22 mg to about 0.32 mg, from about 0.23 mg to about 0.32 mg, from about 0.24 mg to about 0.32 mg, from about 0.20 mg to about 0.30 mg, from about 0.20 mg to about 0.28 mg, from about 0.40 mg to about 0.64 mg, from about 0.42 mg to about 0.64 mg, from about 0.44 mg to about 0.64 mg, from about 0.46 mg to about 0.64 mg, from about 0.48 mg to about 0.64 mg, from about 0.4 mg to about 0.60 mg, from about 0.40 mg to about 0.56 mg, from about 0.48 mg to about 0.56 mg; from about 0.80 mg to about 1.28 mg, from about 0.84 mg to about 1.28 mg, from about 0.88 mg to about 1.28 mg, from about 0.92 mg to about 1.28 mg, from about 0.96 mg to about 1.28 mg, from about 0.8 mg to about 1.20 mg, from about 0.80 mg to about 1.12 mg, from about 0.96 mg to about 1.12 mg; from about 1.2 mg to about 2.0 mg, from about 1.2 mg to about 1.85 mg, from about 1.2 mg to about 1.7 mg, from about 1.2 mg to about 1.65 mg, from about 1.3 mg to about 1.65 mg, from about 1.4 mg to about 1.65 mg, from about 1.45 mg to about 1.70 mg, from about 1.5 mg to about 1.65 mg; from about 1.5 mg to about 2.6 mg, from about 1.6 mg to about 2.6 mg, from about 1.7 mg to about 2.6 mg, from about 1.8 mg to about 2.6 mg, from about 1.8 mg to about 2.5 mg, from about 1.8 mg to about 2.4 mg, from about 1.8 mg to about 2.3 mg, from about 1.8 mg to about 2.20 mg, from about 2.0 mg to about 2.20 mg, from about 2.75 mg to about 4.0 mg, from about 2.75 mg to about 3.75 mg, from about 2.75 mg to about 3.5 mg, from about 3.0 mg to about 3.5 mg, from about 3.25 mg to about 3.75 mg, from about 3.5 mg to about 5.5 mg, from about 3.75 mg to about 5.5 mg, from about 4.0 mg to about 5.0 mg, from about 4.0 mg to about 4.75 mg, from about 4.5 mg to about 6.0 mg, from about 5.0 mg to about 6.0 mg, from about 5.0 mg to about 5.75 mg, or from about 5.0 mg to about 5.5 mg.

Various non-limiting embodiments of a pharmaceutical oral solid dosage form according to the present disclosure can comprise one or more glidants. Glidants may be included in the pharmaceutical oral solid dosage forms, for example, to enhance flowability of the powder ingredients by reducing interparticle friction, particle surface charge, and/or particle cohesion. Non-limiting examples of suitable glidants that can be used in oral solid dosage forms according to the present disclosure include talc, silicon dioxide, colloidal silicon dioxide, and combinations of any thereof.

The concentration of glidant in a pharmaceutical oral solid dosage form according to certain non-limiting embodiments of the present disclosure can range from about 0.6% to about 1%, from about 0.65% to about 1%, from about 0.6% to about 0.95%, from about 0.7% to about 1%, from about 0.7% to about 0.95%, from about 0.75% to about 1%, from about 0.75% to about 0.95%, from about 0.75% to about 0.90%, from about 0.7% to about 0.95%, from about 0.7% to about 0.85%, or from about 0.75% to about 0.85%, all in weight percent based on total weight of the pharmaceutical oral solid dosage form.

In certain non-limiting embodiments, the amount of glidant included in a pharmaceutical oral solid dosage form according to the present disclosure can range from about 0.20 mg to about 0.32 mg, from about 0.21 mg to about 0.32 mg, from about 0.22 mg to about 0.32 mg, from about 0.23 mg to about 0.32 mg, from about 0.24 mg to about 0.32 mg, from about 0.20 mg to about 0.30 mg, from about 0.20 mg to about 0.28 mg, from about 0.40 mg to about 0.64 mg, from about 0.42 mg to about 0.64 mg, from about 0.44 mg to about 0.64 mg, from about 0.46 mg to about 0.64 mg, from about 0.48 mg to about 0.64 mg, from about 0.4 mg to about 0.60 mg, from about 0.40 mg to about 0.56 mg, from about 0.48 mg to about 0.56 mg, from about 0.80 mg to about 1.28 mg, from about 0.84 mg to about 1.28 mg, from about 0.88 mg to about 1.28 mg, from about 0.92 mg to about 1.28 mg, from about 0.96 mg to about 1.28 mg, from about 0.8 mg to about 1.20 mg, from about 0.80 mg to about 1.12 mg, from about 0.96 mg to about 1.12 mg, from about 1.2 mg to about 2.0 mg, from about 1.2 mg to about 1.85 mg, from about 1.2 mg to about 1.7 mg, from about 1.2 mg to about 1.65 mg, from about 1.3 mg to about 1.65 mg, from about 1.4 mg to about 1.65 mg, from about 1.45 mg to about 1.70 mg, from about 1.5 mg to about 1.65 mg; from about 1.5 mg to about 2.6 mg, from about 1.6 mg to about 2.6 mg, from about 1.7 mg to about 2.6 mg, from about 1.8 mg to about 2.6 mg, from about 1.8 mg to about 2.5 mg, from about 1.8 mg to about 2.4 mg, from about 1.8 mg to about 2.3 mg, from about 1.8 mg to about 2.20 mg, from about 2.0 mg to about 2.20 mg, from about 2.75 mg to about 4.0 mg, from about 2.75 mg to about 3.75 mg, from about 2.75 mg to about 3.5 mg, from about 3.0 mg to about 3.5 mg, from about 3.25 mg to about 3.75 mg, from about 3.5 mg to about 5.5 mg, from about 3.75 mg to about 5.5 mg, from about 4.0 mg to about 5.0 mg, from about 4.0 mg to about 4.75 mg, from about 4.5 mg to about 6.0 mg, from about 5.0 mg to about 6.0 mg, from about 5.0 mg to about 5.75 mg, or from about 5.0 mg to about 5.5 mg.

Various non-limiting embodiments of a pharmaceutical oral solid dosage form according to the present disclosure can comprise one or more disintegrant. Disintegrants may be included in the pharmaceutical oral solid dosage forms, for example, to facilitate disintegration after oral administration. Non-limiting examples of suitable disintegrants that can be used in pharmaceutical oral solid dosage forms according to the present disclosure include sodium starch glycolate and/or croscarmellose sodium.

Various non-limiting embodiments of a pharmaceutical oral solid dosage form according to the present disclosure optionally can comprise one or more antioxidant. Examples of antioxidants that can be used in the oral dosage form according to the present disclosure include, but are not limited to, butylated hydroxyanisole (BHA), butylated hydroxytoluene (BHT), ethylenediaminetetraacetic acid (EDTA), sodium metabisulfite, ascorbic acid, citric acid, ascorbyl palmitate, sodium isoascorbate, dihydroguaretic acid, potassium sorbate, sodium bisulfate, sorbic acid, potassium ascorbate, vitamin E, 4-chloro-2,6-diteriarybutylphenol, alphatocopherol, propylgallate, and combinations of two or more thereof.

Various non-limiting embodiments of a pharmaceutical oral solid dosage form according to the present disclosure optionally can comprise one or more coloring agents. Examples of a coloring agent that may be used in oral dosage forms according to the present disclosure include, but are not limited to, FD&C Blue #1 Aluminum Lake, D&C Yellow #10 Aluminum Lake, FD&C Red #40 Aluminum Lake, FD&C Yellow #6 Aluminum Lake, D&C Red #27 Aluminum Lake, D&C Red #30 Helendon Pink Aluminum Lake, FD&C Blue #2 Aluminum Lake.

Various non-limiting embodiments of a pharmaceutical oral solid dosage form according to the present disclosure optionally can further comprise one or more coatings. Coatings may be included on the pharmaceutical oral solid dosage forms to, for example, mask taste, facilitate swallowing, and/or protect active pharmaceutical ingredients. Non-limiting examples of suitable coatings that can be applied to various non-limiting embodiments of the pharmaceutical oral solid dosage forms according to the present disclosure include OPADRY® white coating and OPADRY® II 85F19316 clear coating.

In various non-limiting embodiments, the pharmaceutical oral solid dosage forms according to the present disclosure may be provided as tablets, caplets, capsules, powders, lozenges, or other suitable solid dosage forms.

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, a disintegrant, and a coating. In various embodiments, the pharmaceutical oral solid dosage form is prepared by a method comprising a dry blending process. In various embodiments, the pharmaceutical oral solid dosage form retains at least 90% of an initial levothyroxine salt content in the solid dosage form when the dosage form is stored under one or more of the following storage conditions: stored for nine months under standard testing conditions (25° C.±2° C. and 60%+5% relative humidity); stored for nine months under refrigerated conditions (2° C.±8° C.); stored for 6 months under accelerated testing conditions (40° C.±2° C. and 75%±5% relative humidity); stored for 15 days under extreme testing conditions (60° C.±2° C.).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, a disintegrant, and a coating. In various embodiments, the pharmaceutical oral solid dosage form is prepared by a method comprising a dry blending process. In various embodiments, the pharmaceutical oral solid dosage form retains at least 90% of an initial liothyronine salt content in the solid dosage form when the dosage form is stored under one or more of the following storage conditions: stored for nine months under standard testing conditions (25° C.±2° C. and 60%±5% relative humidity); stored for nine months under refrigerated conditions (2° C.±8° C.); stored for 6 months under accelerated testing conditions (40° C.±2° C. and 75%±5% relative humidity); stored for 15 days under extreme testing conditions (60° C.±2° C.).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, a disintegrant, and a coating. In various non-limiting embodiments, the pharmaceutical oral solid dosage form is prepared by a method comprising a dry blending process. In various embodiments, the pharmaceutical oral solid dosage form retains at least 95% of an initial levothyroxine salt content in the solid dosage form when the dosage form is stored under one or more of the following storage conditions: stored for nine months under standard testing conditions (25° C.±2° C. and 60%±5% relative humidity); stored for nine months under refrigerated conditions (2° C.±8° C.); stored for 6 months under accelerated testing conditions (40° C.±2° C. and 75%±5% relative humidity); stored for 15 days under extreme testing conditions (60° C.±2° C.).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, a disintegrant, and a coating. In various non-limiting embodiments, the pharmaceutical oral solid dosage form is prepared by a method comprising a dry blending process. In various embodiments, the pharmaceutical solid dosage form retains at least 95% of an initial liothyronine salt content in the solid dosage form when the dosage form is stored under one or more of the following storage conditions: stored for nine months under standard testing conditions (25° C.±2° C. and 60%±5% relative humidity); stored for nine months under refrigerated conditions (2° C.±8° C.); stored for 6 months under accelerated testing conditions (40° C.±2° C. and 75%±5% relative humidity); stored for 15 days under extreme testing conditions (60° C.±2° C.).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, a disintegrant, and a coating. In various non-limiting embodiments, the pharmaceutical oral solid dosage form is prepared by a method comprising a dry blending process. In various embodiments, the pharmaceutical oral solid dosage form retains at least 99% of an initial levothyroxine content in the solid dosage form when the dosage form is stored under one or more of the following storage conditions: stored for nine months under standard testing conditions (25° C.±2° C. and 60%±5% relative humidity); stored for nine months under refrigerated conditions (2° C.±8° C.); stored for 6 months under accelerated testing conditions (40° C.±2° C. and 75%±5% relative humidity); stored for 15 days under extreme testing conditions (60° C.±2° C.).

In certain embodiments according to the present disclosure, a pharmaceutical oral solid dosage form prepared by a dry uniform blending process comprises levothyroxine salt, liothyronine salt, microcrystalline cellulose, and at least one excipient selected from a lubricant, a glidant, a diluent, a disintegrant, and a coating. In various non-limiting embodiments, the pharmaceutical oral solid dosage form is prepared by a method comprising a dry blending process. In various embodiments, the pharmaceutical oral solid dosage form retains at least 99% of an initial liothyronine content in the solid dosage form when the dosage form is stored for nine months under standard testing conditions (25° C.±2° C. and 60%±5% relative humidity) and/or nine months under refrigerated conditions (2° C.±8° C.) and/or 6 months under accelerated testing conditions (40° C.±2° C. and 75%±5% relative humidity) and/or 15 days under extreme testing conditions (60° C.±2° C.).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, an antioxidant, and a coloring agent. In various embodiments, the pharmaceutical oral solid dosage form is prepared by a method comprising a dry blending process. In various embodiments, the pharmaceutical oral solid dosage form retains at least 90% of an initial levothyroxine salt content in the solid dosage form when the dosage form is stored under one or more of the following storage conditions: stored for 6 months under standard testing conditions (25° C.±2° C. and 60%±5% relative humidity); stored for 6 months under accelerated testing conditions (40° C.±2° C. and 75%±5% relative humidity).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, an antioxidant, and a coloring agent. In various embodiments, the pharmaceutical oral solid dosage form is prepared by a method comprising a dry blending process. In various embodiments, the pharmaceutical oral solid dosage form retains at least 90% of an initial liothyronine salt content in the solid dosage form when the dosage form is stored under one or more of the following storage conditions: stored for 6 months under standard testing conditions (25° C.±2° C. and 60%±5% relative humidity); stored for 6 months under accelerated testing conditions (40° C.±2° C. and 75%±5% relative humidity).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, an antioxidant, and a coloring agent. In various embodiments, the pharmaceutical oral solid dosage form is prepared by a method comprising a dry blending process. In various embodiments, the pharmaceutical oral solid dosage form retains at least 95% of an initial levothyroxine salt content in the solid dosage form when the dosage form is stored under one or more of the following storage conditions: stored for 6 months under standard testing conditions (25° C.±2° C. and 60%±5% relative humidity); stored for 6 months under accelerated testing conditions (40° C.±2° C. and 75%±5% relative humidity).

In certain embodiments according to the present disclosure, a pharmaceutical oral solid dosage form comprises levothyroxine salt, liothyronine salt, microcrystalline cellulose, and at least one excipient selected from a lubricant, a glidant, a diluent, an antioxidant, and a coloring agent. In various embodiments, the pharmaceutical oral solid dosage form is prepared by a method comprising a dry blending process. In various embodiments, the pharmaceutical oral solid dosage form retains at least 95% of an initial liothyronine salt content in the solid dosage form when the dosage form is stored under one or more of the following storage conditions: stored for 6 months under standard testing conditions (25° C.±2° C. and 60%±5% relative humidity); stored for 6 months under accelerated testing conditions (40° C.±2° C. and 75%±5% relative humidity).

In various embodiments, pharmaceutical oral solid dosage forms according to the present disclosure can be prepared by a method comprising a dry blending process that provides for uniform blending of ingredients. The dry uniform blending of the ingredients can comprise techniques known in the art, such as one or more of blending, mixing, sieving, co-milling, grinding, granulation, and power granulation.

In certain non-limiting embodiments according to the present disclosure, a prepackaged drug product is provided comprising pharmaceutical oral solid dosage forms according to the present disclosure contained in a container closure system including a container and a sealing closure. In certain non-limiting embodiments according to the present disclosure, the container has an internal volume of about 15 cm³ to about 45 cm³ and includes therein about 10 to about 20 oral solid dosage forms according to the present disclosure and, optionally, at least one silica gel desiccant canister containing, for example, about 2 grams of desiccant per canister. In various embodiments, the container closure system can also include at least one oxygen absorber containing, for example, about 2 grams of oxygen absorbing material. In certain non-limiting embodiments according to the present disclosure, a prepackaged drug product according to the present disclosure comprises a container closure system wherein the container has an internal volume of about 15 cm³ to about 45 cm³ and in which are contained about 10 to about 20 oral solid dosage forms according to the present disclosure, at least one silica gel desiccant canister (including, for example, about 2 grams of desiccant per canister), and at least one oxygen absorber (including, for example, about 2 grams of oxygen absorbing material).

In other non-limiting embodiments, a prepackaged drug product is provided comprising oral solid dosage forms according to the present disclosure contained in a container closure system including a container and a sealing closure. In certain non-limiting embodiments according to the present disclosure, the container has an internal volume of about 60 cm³ to about 90 cm³ and includes therein about 90 to about 110 oral solid dosage forms according to the present disclosure and, optionally, at least one silica gel desiccant canister containing, for example, about 2 grams of desiccant per canister. In various embodiments, the container closure system can also include therein at least one oxygen absorber containing, for example, about 2 grams of oxygen absorbing material. In certain non-limiting embodiments according to the present disclosure, a prepackaged drug product according to the present disclosure comprises a container closure system wherein the container has an internal volume of about 60 cm³ to about 90 cm³ and in which are contained about 90 to about 110 oral solid dosage forms according to the present disclosure, at least one silica gel desiccant canister (including, for example, about 2 grams of desiccant per canister), and at least one oxygen absorber (including, for example, about 2 grams of oxygen absorbing material).

The container of the container closure system of certain non-limiting embodiments of a prepackaged drug product according to the present disclosure can comprise at least one of glass, plastic, or other suitable polymer material. For example, in certain embodiments the container of the container closure system of certain non-limiting embodiments of a prepackaged drug product according to the present disclosure can comprise polypropylene or high-density polyethylene. In certain embodiments of a prepackaged drug product according to the present disclosure, the pharmaceutical oral solid dosage forms can be stored in a container of a container closure system that has been purged with an inert gas to replace at least a portion of environmental oxygen within the container prior to sealing the container with the closure. In certain embodiments according to the present disclosure, the inert gas used to purge the container can comprise nitrogen, argon, a mixture of nitrogen and argon, or another inert gas. In certain embodiments of a prepackaged drug product according to the present disclosure, the pharmaceutical oral solid dosage forms can be stored in a container of a container closure system that has not been purged with an inert gas prior to sealing the container with the closure. In certain embodiments according to the present disclosure, at least a portion of air space within the container may be filled with a pill packing material such as, for example, rayon or cotton, prior to sealing the container with the closure.

An aspect of the present disclosure is directed to methods of administering a pharmaceutical oral solid dosage form comprising levothyroxine and liothyronine according to the present disclosure to a patient or human subject in need thereof. According to one method, a pharmaceutical oral solid dosage form according to the present disclosure is administered to a patient or human subject in need thereof such that that the patient/subject receives about 7.5 μg to about 300 μg of levothyroxine and about 1.75 μg to about 75 μg of liothyronine per dosage.

According to certain non-limiting embodiments, pharmaceutical oral solid dosage forms according to the present disclosure may be prepared using a dry layering/mixing process generally including the following steps. A dry layered blend is prepared by first screening a diluent (e.g., AVICEL® HFE-102 powder or MCC) through a #30 mesh screen and then mixing the diluent using a high shear mixer granulator (HSMG) to form a first layer in the HSMG. Next, a screened blend is prepared by screening together the remaining diluent, additional diluent (e.g. butylhydroxyanisole, mannitol, hydroxypropyl methylcellulose), a glidant (e.g., talc), and active pharmaceutical ingredients (levothyroxine sodium, liothyronine sodium) to form a screened blend. The screened blend is deposited on the first layer to form a second layer in the HSMG. Next, a dry lubricant (e.g. magnesium stearate) is screened through a #30 mesh screen and added to the mixed and screened dry blend, and the combined ingredients are uniformly mixed in the HSMG to form a substantially uniform dry pharmaceutical mixture. Predetermined quantities of the dry mixture are compressed to form tableted dosage forms having a desired mass and including desired quantities of levothyroxine and liothyronine salts per tablet. Alternatively, predetermined quantities of the dry pharmaceutical mixture may be, for example, disposed in capsules such as, for example, hard gelatin capsules, or used to prepare other suitable pharmaceutical oral solid dosage forms.

According to certain non-limiting embodiments, pharmaceutical oral solid dosage forms according to the present disclosure may be prepared using a dry layering/mixing process generally including the following steps. A dry layered blend is prepared by first screening a diluent (e.g., AVICEL® PH-102 MCC powder) through a #30 mesh screen and then mixing the diluent using a high shear mixer granulator (HSMG) to form a first layer in the HSMG. Next, a screened blend is prepared by screening together the remaining diluent, additional diluent (e.g. butylhydroxyanisole, mannitol), a glidant (e.g., talc), and active pharmaceutical ingredients (levothyroxine sodium, liothyronine sodium) to form a screened blend. The screened blend is deposited on the first layer to form a second layer in the HSMG. Next, a dry lubricant (e.g. magnesium stearate) is screened through a #30 mesh screen and added to the mixed and screened dry blend, and the combined ingredients are uniformly mixed in the HSMG to form a substantially uniform dry pharmaceutical mixture. Predetermined quantities of the dry mixture are compressed to form tableted dosage forms having a desired mass and including desired quantities of levothyroxine and liothyronine salts per tablet. Alternatively, predetermined quantities of the dry pharmaceutical mixture may be, for example, disposed in capsules such as, for example, hard gelatin capsules, or used to prepare other suitable pharmaceutical oral solid dosage forms

The present inventors observed that embodiments of pharmaceutical oral solid dosage forms according to the present disclosure including levothyroxine and liothyronine salts exhibit advantageous stability. For example, the present inventors observed that certain non-limiting embodiments of pharmaceutical oral solid dosage forms according to the present disclosure retained at least 90% of an initial amount of levothyroxine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 90% of an initial amount of levothyroxine salt after storage for one month, two months, three months, or six months under accelerated testing conditions including a temperature of about 38° C. to about 42° C. and a relative humidity from about 70% to about 80%. The present inventors also observed that certain non-limiting embodiments of pharmaceutical oral solid dosage forms according to the present disclosure retained at least 90% of an initial amount of levothyroxine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 90% of an initial amount of levothyroxine salt after storage for 15 days at a temperature of about 58° C. to about 62° C.

The present inventors observed that certain non-limiting embodiments of pharmaceutical oral solid dosage forms according to the present disclosure retained at least 92% of an initial amount of levothyroxine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 92% of an initial amount of levothyroxine salt after storage for one month, two months, three months, or six months under accelerated testing conditions including a temperature of about 38° C. to about 42° C. and a relative humidity from about 70% to about 80%. The present inventors also observed that certain non-limiting embodiments of pharmaceutical oral solid dosage forms according to the present disclosure retained at least 92% of an initial amount of levothyroxine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 92% of an initial amount of levothyroxine salt after storage for 15 days at a temperature of about 58° C. to about 62° C.

The present inventors observed that certain non-limiting embodiments of pharmaceutical oral solid dosage forms according to the present disclosure retained at least 95% of an initial amount of levothyroxine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 95% of an initial amount of levothyroxine salt after storage for one month, two months, three months, or six months under accelerated testing conditions including a temperature of about 38° C. to about 42° C. and a relative humidity from about 70% to about 80%. The present inventors also observed that certain non-limiting embodiments of pharmaceutical oral solid dosage forms according to the present disclosure retained at least 95% of an initial amount of levothyroxine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 95% of an initial amount of levothyroxine salt after storage for 15 days at a temperature of about 58° C. to about 62° C.

The present inventors observed that certain non-limiting embodiments of pharmaceutical oral solid dosage forms according to the present disclosure retained at least 90% of an initial amount of liothyronine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 90% of an initial amount of liothyronine salt after storage for one month, two months, three months, or six months under accelerated testing conditions including a temperature of about 38° C. to about 42° C. and a relative humidity from about 70% to about 80%. The present inventors also observed that certain non-limiting embodiments of pharmaceutical oral solid dosage forms according to the present disclosure retained at least 90% of an initial amount of liothyronine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 90% of an initial amount of liothyronine salt after storage for 15 days at a temperature of about 58° C. to about 62° C.

The present inventors observed that certain non-limiting embodiments of pharmaceutical oral solid dosage forms according to the present disclosure retained at least 92% of an initial amount of liothyronine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 92% of an initial amount of liothyronine salt after storage for one month, two months, three months, or six months under accelerated testing conditions including a temperature of about 38° C. to about 42° C. and a relative humidity from about 70% to about 80%. The present inventors also observed that certain non-limiting embodiments of pharmaceutical oral solid dosage forms according to the present disclosure retained at least 92% of an initial amount of liothyronine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 92% of an initial amount of liothyronine salt after storage for 15 days at a temperature of about 58° C. to about 62° C.

The present inventors observed that certain non-limiting embodiments of pharmaceutical oral solid dosage forms according to the present disclosure retained at least 95% of an initial amount of liothyronine salt after storage for one month, two months, three months, six months, or nine months under standard testing conditions including a temperature of about 23° C. to about 27° C. and a relative humidity from about 55% to about 65%. The present inventors also observed that certain embodiments of pharmaceutical oral solid dosage forms according to the present disclosure retained at least 95% of an initial amount of liothyronine salt after storage for one month, two months, three months, or six months under accelerated testing conditions including a temperature of about 38° C. to about 42° C. and a relative humidity ranging from about 70% to about 80%. The present inventors also observed that certain non-limiting embodiments of pharmaceutical oral solid dosage forms according to the present disclosure retained at least 95% of an initial amount of liothyronine salt after storage for one month, three months, six months, or nine months under refrigerated testing conditions including a temperature of about 2° C. to about 8° C. The present inventors also observed that certain non-limiting embodiments of pharmaceutical oral solid dosage forms according to the present disclosure retained at least 95% of an initial amount of liothyronine salt after storage for 15 days at a temperature of about 58° C. to about 62° C.

Following are non-limiting examples of pharmaceutical oral dosage forms and methods of making pharmaceutical oral dosage forms according to the present disclosure.

### EXAMPLES

### Example 1

This example describes a non-limiting embodiment of a dry layering/mixing and tableting process that may be used to make embodiments of pharmaceutical oral solid dosage forms according to the present disclosure.

Twenty-five percent of a total required amount of AVICEL® HFE-102 material was screened through a #30 mesh screen, and the screened material was then mixed in a high shear mixer granulator (HSMG) (Glatt VG PRO) for thirty seconds at a power setting of 5 (500 rpms per minute) with amperage at 3-4 amperes. The screened and mixed portion of the AVICEL® HFE-102 material formed a first layer in the HSMG.

Next, a screened blend was prepared by dry screening through a #30 mesh screen into a vessel the remaining 75% of the total needed amount of AVICEL® HFE 102 material, levothyroxine sodium (previously screened through a #60 mesh screen), liothyronine sodium (previously screened through a #200 mesh screen), butylhydroxyanisole (previously screened through a #200 mesh screen), mannitol (previously screened through a #60 mesh screen), hydroxypropyl methylcellulose (HPMC) K-100 LV CR LH and talc (previously screened through a #30 mesh screen). The screened levothyroxine sodium and liothyronine sodium were sandwiched in the HSMG between the layers of screened AVICEL® HFE 102. Screened butylhydroxyanisole was then added, followed by screened mannitol, and screened talc and HPMC K-100 LV CR LH. This screened blend was positioned in the HSMG to form a second layer adjacent to the first (AVICEL® HFE-102 material) layer.

The assembly of two layers disposed in the HSMG constituted a dry layered blend. The HSMG was activated to uniformly mix the dry layered blend for eight minutes using an HSMG power setting of 5 (500 rpms per minute) with amperage at 3-4 amperes. The HSMG was operated under low purge air pressure of 0.02 psi. The mixed dry layered blend was removed from the HSMG and screened through a #30 mesh screen and returned to the HSMG for further uniform mixing for 8 minutes using an HSMG power setting of 5 (500 rpm per minute) with amperage at 3-4 amperes.

Magnesium stearate was screened through a #30 mesh screen and added to the doubly mixed and screened dry layered blend, and the materials were uniformly mixed for 30 seconds using an HSMG power setting of 5 (500 rpms per minute) with amperage at 3-4 amperes to form a final oral dosage mixture.

Portions of the final oral dosage mixture were removed from the HSMG using a cradle discharge method. The cradle discharge method removed multiple small quantities of the final oral dosage mixture from the HSMG over short time periods to prevent segregation of the ingredients. Finally, measured portions of the dry oral dosage mixture were compressed into 130 mg tablets using a Piccola/Globe pharma tablet press. These were compressed to a hardness of 4-8 Kp.

The tablets were then packaged in either 30 cm³ or 75 cm³ high density polyethylene bottles.

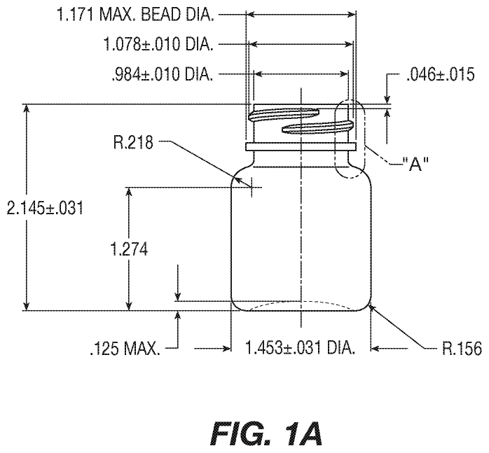

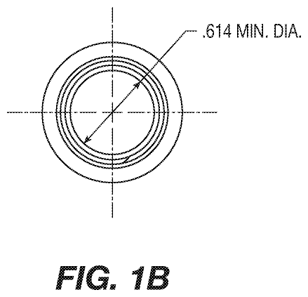

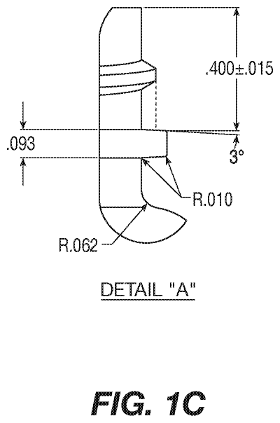

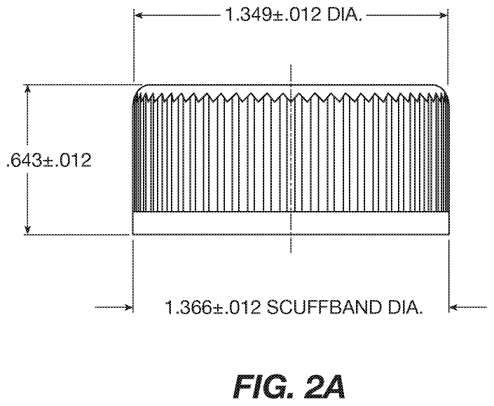

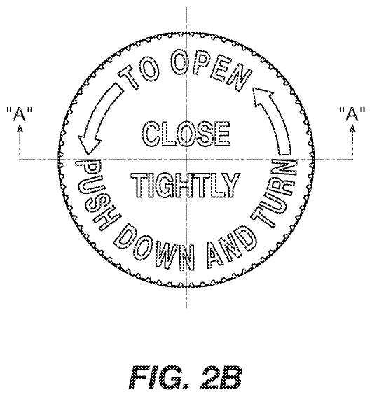

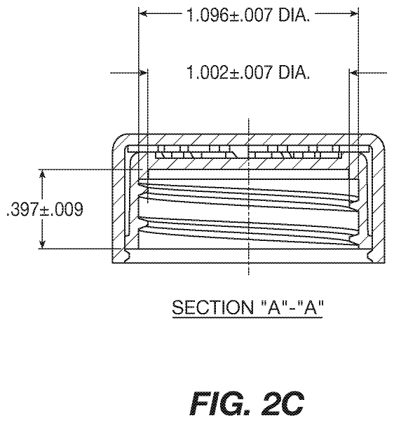

About 10 to about 20 of the tablets and 0, 1, or 2 silica gel desiccant canisters (commercially available from, e.g., Clariant International, Colton, CA) (each canister including about 2 g of desiccant) were disposed in the 30 cm³ HDPE containers (shown in FIGS. 1A, 1B, and 1C). In certain of the 30 cm³ containers including about 10 to 20 tablets, 0, 1, or 2 PHARMAKEEP® oxygen absorber canisters (commercially available from Clariant International) (each canister including about 2 g of oxygen absorber) also were disposed. The 30 cm³ containers were sealed with a sealing closure (depicted in FIGS. 2A, 2B, and 2C), providing a packaged pharmaceutical oral solid dosage form product.

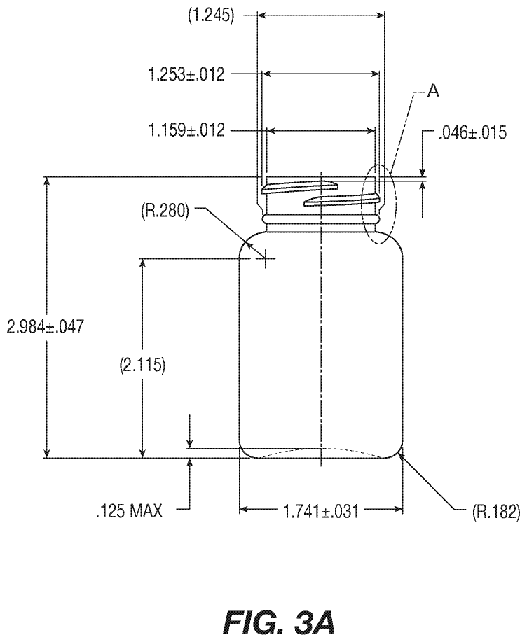

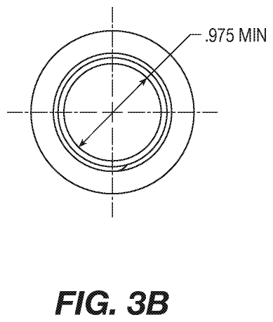

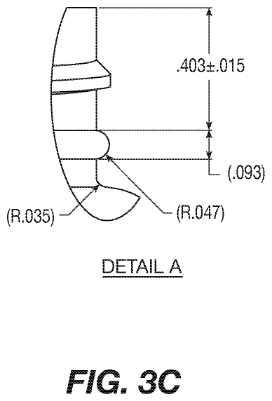

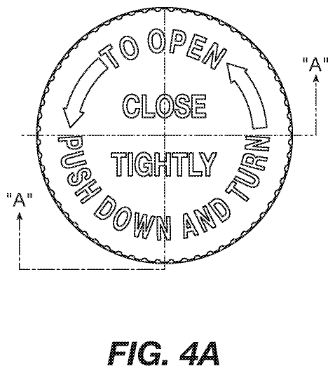

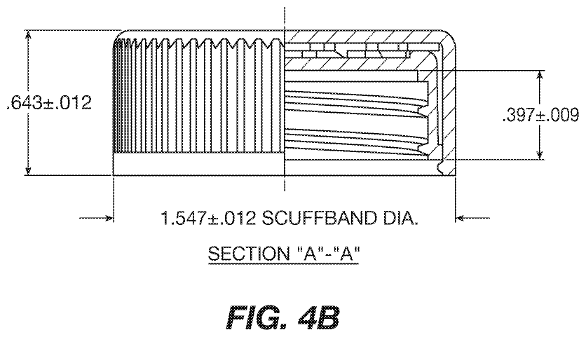

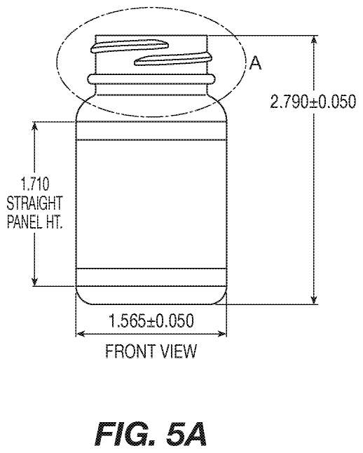

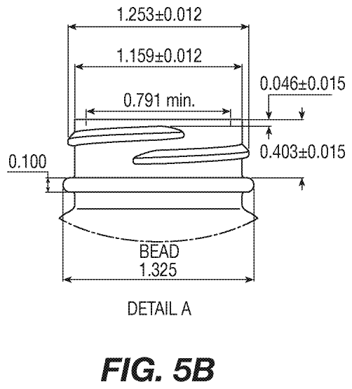

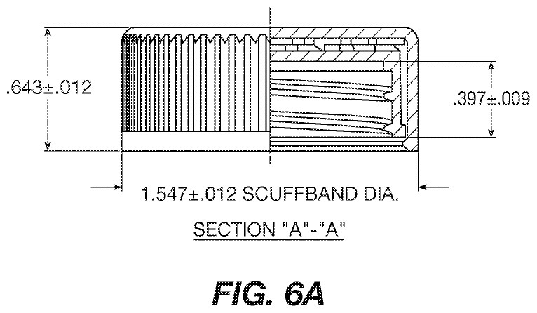

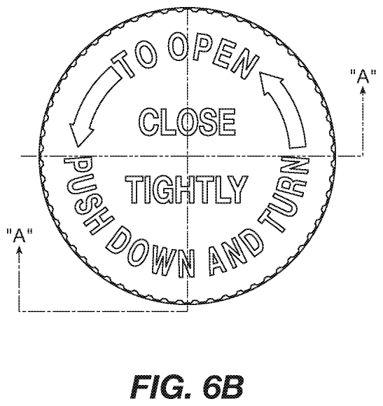

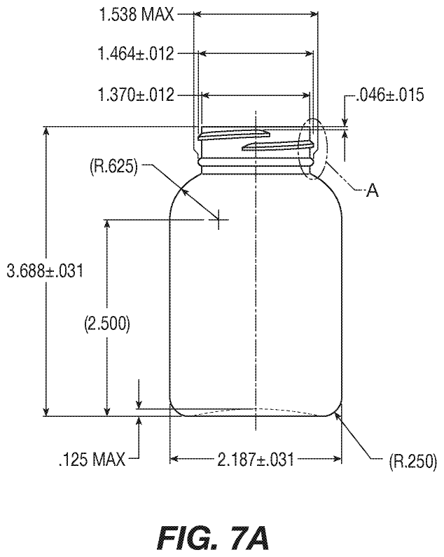

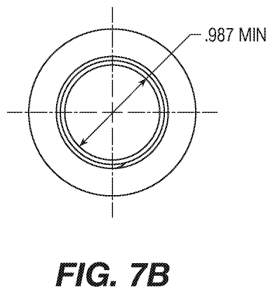

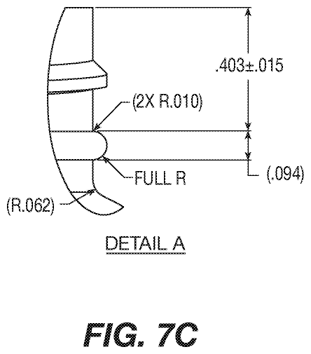

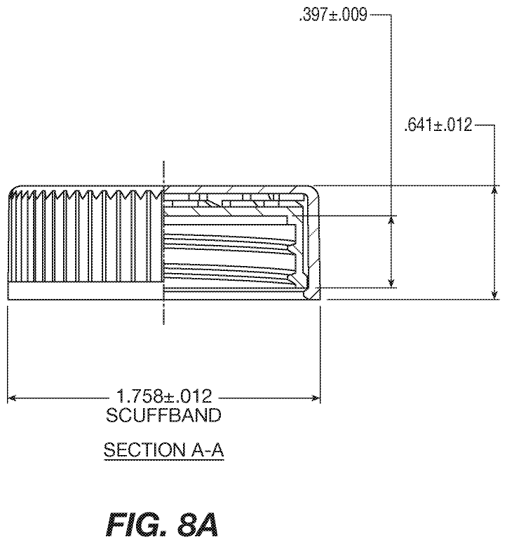

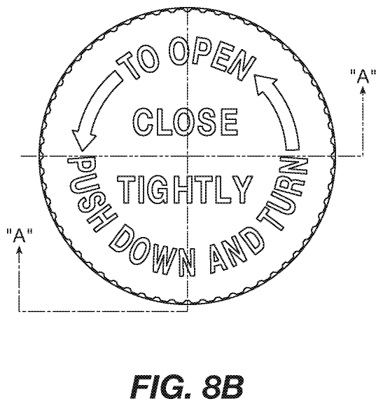

About 90 to about 110 the tablets and 0, 1, or 2 of the silica gel desiccant canisters were disposed in the 75 cm³ containers (shown in FIGS. 3A, 3B, and 3C). In certain of the 75 cm³ containers including about 90 to 110 tablets, 0, 1, or 2 of the PHARMAKEEP® oxygen absorber canisters also were disposed. The 75 cm³ containers were sealed with a sealing closure (shown in FIGS. 4A and 4B), providing a packaged pharmaceutical oral solid dosage form product.

### Example 2

Tablets individually including about 2.25 μg to about 62.5 μg liothyronine sodium and about 9.5 μg to about 250 μg levothyroxine sodium were prepared by compressing portions of several uniform mixtures made using the method generally described in Example 1. Each of the tablets included liothyronine sodium, levothyroxine sodium, microcrystalline cellulose (MCC), mannitol, hydroxypropyl methylcellulose, butylhydroxyanisole, talc, and magnesium stearate. Table 1 below provides the mass of the ingredients in individual tablets comprising about 2.25 μg to about 62.5 μg liothyronine sodium and about 9.5 μg to about 250 levothyroxine sodium per tablet, which contents are referred to as the “strength” of the individual tablets.

TABLE 1
Composition of tablets

| | All Tablet Strengths | | | | | | | | | | |
|---|---|---|---|---|---|---|---|---|---|---|---|
| | 2.25/9.5 | 3.1/12.5 | 4.5/19 | 6.25/25 | 9/38 | 12.5/50 | 13.5/57 | 18/76 | 25/100 | 37.5/150 | 62.5/250 |
| Ingredient | mg/tablet | mg/tablet | mg/tablet | mg/tablet | mg/tablet | mg/tablet | mg/tablet | mg/tablet | mg/tablet | mg/tablet | mg/tablet |
| Liothyronine | 0.00225 | 0.0031 | 0.0045 | 0.00625 | 0.009 | 0.0125 | 0.0135 | 0.018 | 0.025 | 0.0375 | 0.0625 |
| sodium, USP | | | | | | | | | | | |
| Levothyroxine | 0.0095 | 0.0125 | 0.019 | 0.025 | 0.038 | 0.05 | 0.057 | 0.076 | 0.1 | 0.15 | 0.25 |
| sodium, USP | | | | | | | | | | | |
| Avicel ® HFE | 107.203 | 107.039 | 107.261 | 107.114 | 107.178 | 107.132 | 107.094 | 106.941 | 107.090 | 106.937 | 107.032 |
| 102 Powder | | | | | | | | | | | |
| Mannitol, USP | 14.65 | 14.65 | 14.65 | 14.65 | 14.65 | 14.65 | 14.65 | 14.65 | 14.65 | 14.65 | 14.65 |
| Hydroxypropyl | 6.00 | 6.00 | 6.00 | 6.00 | 6.00 | 6.00 | 6.00 | 6.00 | 6.00 | 6.00 | 6.00 |
| methylcellulose, | | | | | | | | | | | |
| NF | | | | | | | | | | | |
| Butylhydroxy- | 0.005 | 0.005 | 0.005 | 0.005 | 0.005 | 0.005 | 0.005 | 0.005 | 0.005 | 0.005 | 0.005 |
| anisole, NF | | | | | | | | | | | |
| Talc, NF | 1.00 | 1.00 | 1.00 | 1.00 | 1.00 | 1.00 | 1.00 | 1.00 | 1.00 | 1.00 | 1.00 |
| Magnesium | 1.00 | 1.00 | 1.00 | 1.00 | 1.00 | 1.00 | 1.00 | 1.00 | 1.00 | 1.00 | 1.00 |
| Stearate, NF | | | | | | | | | | | |
| FD&C Blue #1 | — | 0.160 | 0.06 | — | — | — | 0.09 | 0.16 | 0.05 | — | — |
| Aluminum Lake | | | | | | | | | | | |
| D&C Yellow #10 | 0.130 | — | — | 0.05 | 0.06 | — | 0.09 | 0.15 | 0.07 | 0.2 | — |
| Aluminum Lake | | | | | | | | | | | |
| FD&C Red #40 | — | 0.130 | — | 0.15 | 0.06 | 0.15 | — | — | 0.01 | 0.02 | — |
| Aluminum Lake | | | | | | | | | | | |
| Total | 130.00 | 130.00 | 130.00 | 130.00 | 130.00 | 130.00 | 130.00 | 130.00 | 130.00 | 130.00 | 130.00 |

### Example 3

A study was conducted to assess the stability of certain embodiments of pharmaceutical oral solid dosage forms according to the present disclosure.

Tablets including about 3.1 μg liothyronine sodium and about 12.5 μg levothyroxine sodium were prepared by compressing portions of several uniform mixtures made using the method generally described in Example 1. Each of the mixtures included liothyronine sodium, levothyroxine sodium, MCC, mannitol, hydroxypropyl methylcellulose, butylhydroxyanisole, talc, and magnesium stearate. The MCC was included as a component of Avicel® HFE 102 powder. Table 2 below provides the mass and weight/weight concentration of the ingredients in individual tablets comprising about 3.1 μg liothyronine sodium and about 12.5 μg levothyroxine sodium per tablet produced in Batch NB543-050.

TABLE 2
Composition of tablets in Batch NB543-050

| Ingredient | % w/w | mg/unit |
|---|---|---|
| Liothyronine sodium, USP | 0.00238 | 0.00310 |
| Levothyroxine sodium, USP | 0.00961 | 0.0125 |
| Avicel ® HFE 102 Powder | 84.65 | 110.039 |
| Mannitol, USP | 11.27 | 14.65 |
| Hydroxypropyl | 2.31 | 3.00 |
| methylcellulose, NF | | |
| Butylhydroxyanisole, NF | 0.00384 | 0.005 |
| Talc, NF | 0.77 | 1.00 |
| Magnesium Stearate, NF | 0.77 | 1.00 |
| FD&C Blue #1 Aluminum | 0.12 | 0.160 |
| Lake | | |
| D&C Yellow #10 Aluminum | — | — |
| Lake | | |
| FD&C Red #40 Aluminum | 0.10 | 0.130 |
| Lake | | |
| Total | 100.00 | 130 |

Solid dosage forms including different amounts of the active agents (levothyroxine sodium and liothyronine sodium) may be formed in a dose similar manner by adjusting the amount of the active ingredient, along with the amount of inactive ingredients to keep the mass of the dosage forms the same.

For purposes of conducting the stability study, the tablets of Batch NB543-050 were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed with a closure. As an alternative, air may be displaced within a container by disposing an inert pill packing material, for example, rayon or cotton, in the container after tablet loading and before induction sealing.

Four studies were conducted to assess the stability of the levothyroxine sodium and liothyronine sodium in the tablets produced in Batch NB543-050. The stability studies involved the following testing periods and conditions:
Storing under refrigerated conditions (2° C. to 8° C.) for up to nine months.
Storing under standard testing conditions (25° C.±2° C. and 60%±5% relative humidity) for up to nine months.
Storing under accelerated testing conditions (40° C.±2° C. and 75%±5% relative humidity) for up to six months.
Storing under extreme testing conditions (60° C.±2° C.) for 7 or 15 days.

A refrigerated testing conditions stability study of the tablets of Batch NB543-050 contained in the container closure system was conducted over a period of nine months to determine the rate of physical or chemical degradation of the levothyroxine sodium and liothyronine sodium included in tablets when stored under the refrigerated environmental conditions.

Refrigerated condition stability samples were placed upright in an environmental chamber and maintained in an upright position during the stability testing. As discussed, each tablet was disposed in the sealed container containing either 10 to 20 or 90 to 100 tablets from Batch NB543-050, and each tablet included about 3.1 μg liothyronine sodium and about 12.5 μg levothyroxine sodium per tablet. The sealed containers were maintained at 2° C. to 8° C., uninterrupted, (except for the addition or withdrawal of test samples) for a period of one month, three months, six months, or nine months. Tablets were removed from the storage conditions at the specified time points and tested for levothyroxine sodium and liothyronine sodium contents using high pressure liquid chromatography.

Considering levothyroxine sodium content, shelf life of tablets of Batch NB543-050 was estimated to be at least nine months when stored under refrigerated testing conditions. Table 3 provides the nine-month stability assay results for tablets of Batch NB543-050 stored under refrigerated testing conditions. The stability assay showed that greater than 95 percent of the original levothyroxine sodium content was retained in tablets stored under refrigerated testing conditions for nine months.

Considering liothyronine sodium content, shelf life of tablets of Batch NB543-050 was estimated to be at least nine months when stored under refrigerated testing conditions. As shown in Table 3, the stability assay showed that greater than 95 percent of the original liothyronine sodium content was retained in tablets stored under refrigerated testing conditions for nine months.

A standard testing conditions stability study of the tablets of Batch NB543-050 contained in the container closure system was conducted over a period of nine months to determine the rate of physical or chemical degradation of the levothyroxine sodium and liothyronine sodium included in tablets when stored under the standard environmental conditions.

Container closure systems prepared as described above containing tablets including about 3.1 μg liothyronine sodium and about 12.5 μg levothyroxine sodium per tablet were placed upright in a calibrated environmental chamber and maintained in an upright position during the stability study. The sealed containers including either 10 to 20 or 90 to 100 tablets were maintained at 25° C.±2° C. and 60%±5% relative humidity, uninterrupted (except for the addition or withdrawal of test samples), for a period of one month, two months, three months, six months, or nine months. Tablets were removed from the storage conditions at the specified time points and tested for levothyroxine sodium and liothyronine sodium contents using high pressure liquid chromatography.

Considering levothyroxine sodium content, shelf life of tablets of Batch NB543-050 was estimated to be at least nine months when stored under standard testing conditions. Table 3 provides the nine-month stability assay results for tablets of Batch NB543-050. The stability assay showed that greater than 95 percent of the original levothyroxine sodium content was retained in tablets stored under standard testing conditions for nine months.

Considering liothyronine sodium content, shelf life of tablets of Batch NB543-050 was estimated to be at least nine months when stored under standard testing conditions. As shown in Table 3, the stability assay showed that greater than 95 percent of the original liothyronine sodium content was retained in tablets stored under standard testing conditions for nine months.

Accelerated condition stability samples were placed upright in an environmental chamber and maintained in an upright position during the stability testing. As discussed, each tablet was disposed in the sealed container containing either 10 to 20 or 90 to 100 tablets from Batch NB543-050, and each tablet included about 3.1 μg liothyronine sodium and about 12.5 μg levothyroxine sodium per tablet. The sealed containers were maintained at 40° C.±2° C. and 75%±5% relative humidity, uninterrupted, (except for the addition or withdrawal of test samples) for a period of one month, two months, three months, or six months. Tablets were removed from the storage conditions at the specified time points and tested for levothyroxine sodium and liothyronine sodium contents using high pressure liquid chromatography.

Considering levothyroxine sodium content, shelf life of tablets of Batch NB543-050 was estimated to be at least six months when stored under accelerated testing conditions. Table 3 provides the six-month stability assay results for tablets of Batch NB543-050 stored under accelerated testing conditions. The stability assay showed that greater than 95 percent of the original levothyroxine sodium content was retained in tablets stored under accelerated testing conditions for six months.

Considering liothyronine sodium content, shelf life of tablets of Batch NB543-050 was estimated to be at least six months when stored under accelerated testing conditions. As shown in Table 3, the stability assay showed that greater than 95 percent of the original liothyronine sodium content was retained in tablets stored under accelerated testing conditions for six months.

An extreme testing conditions stability study was conducted in which tablets of Batch NB543-050 were stored under extreme testing conditions for a period of 7 or 15 days to determine the rate of physical or chemical degradation of the original content of about 3.1 μg liothyronine sodium and about 12.5 μg levothyroxine sodium in the tablets. All extreme testing conditions stability samples (each sample comprising a container closure system containing either 10 to 20 or 90 to 100 tablets of Batch NB543-050) were placed upright in an environmental chamber and maintained in an upright position during the stability study. All of the containers were maintained at 60° C.±2° C., uninterrupted (except for the addition or withdrawal of test samples), for a period of 7 or 15 days. Tablets were removed from the storage conditions at the specified time points and tested for levothyroxine sodium and liothyronine sodium content using high pressure liquid chromatography.

Considering levothyroxine sodium content, shelf life of tablets of Batch NB543-050 was estimated to be at least fifteen days when stored under extreme testing conditions. Table 3 provides the fifteen-day stability assay results for tablets of Batch NB543-050. The stability assay showed that greater than 95 percent of the original levothyroxine sodium content was retained in tablets stored under extreme testing conditions for fifteen days.

Considering liothyronine sodium content, shelf life of tablets of Batch NB543-050 was estimated to be at least fifteen days when stored under extreme testing conditions. As shown in Table 3, the stability assay showed that greater than 95 percent of the original liothyronine sodium content was retained in tablets stored under extreme testing conditions for fifteen days.

Table 3 reports stability data for the pharmaceutical oral solid dosage form samples (Batch NB543-050) tested under refrigerated, standard, accelerated, and extreme conditions. The assay percentages listed in Table 2 are based on expected (“label claim”) contents of about 3.1 μg liothyronine sodium and about 12.5 μg levothyroxine sodium per tablet of Batch NB543-050.

TABLE 3
Stability assay results for tablets of Batch NB543-050

Attribute
Assay % Label Claim

| Stability | Levothyroxine | Liothyronine |
|---|---|---|
| Condition | Sodium | Sodium |
| T0 | 103.8 | 98 |
| 7 days/60 °C | 100.8 | 99 |
| 15 days/60 °C | 98.6 | 97.6 |
| 1 M, 2 °C-8 °C | 102.3 | 100.5 |
| 3 M, 2 °C-8 °C | 102.8 | 101.7 |
| 6 M, 2 °C-8 °C | 102.3 | 100.9 |
| 9 M, 2 °C-8 °C | 103.9 | 101.3 |
| 1 M, 25 °C/60% RH | 100.5 | 99.6 |
| 2 M, 25 °C/60% RH | 102.2 | 100.7 |
| 3 M, 25 °C/60% RH | 103.1 | 101.8 |
| 6 M, 25 °C/60% RH | 102.6 | 101.1 |
| 9 M, 25 °C/60% RH | 102.5 | 100.3 |
| 1 M, 40 °C/75% RH | 100.5 | 99.8 |
| 2 M, 40 °C/75% RH | 100.6 | 98.5 |
| 3 M, 40 °C/75% RH | 101.3 | 101.0 |
| 6 M, 40 °C/75% RH | 101.5 | 98.7 |

With reference to the results shown in Table 3, the present inventors surprisingly observed that pharmaceutical oral solid dosage forms produced in Batch NB543-050 retained 100% of the original content of levothyroxine sodium when stored under refrigerated testing conditions at 2° C. to 8° C. for a period of nine months, and also retained about 99% of the original content of levothyroxine sodium when stored under standard testing conditions at 25° C.±2° C. and 60%±5% relative humidity for a period of nine months. Even when tablets were stored under accelerated conditions (40° C.±2° C. and 75%±5% relative humidity) for six months, tablets produced in Batch NB543-050 retained about 98% of the original levothyroxine sodium content (a decrease in the levothyroxine sodium content of the tablets of about two percent). When tablets were stored under extreme conditions (60° C.±2° C.) for 15 days, tablets produced in Batch NB543-050 retained about 95% of the original levothyroxine sodium content.

With reference to the results shown in Table 3, the present inventors also surprisingly observed that pharmaceutical oral solid dosage forms produced in Batch NB543-050 retained 100% of the original content of liothyronine sodium when stored under refrigerated testing conditions at 2° C. to 8° C. for a period of nine months, and also retained 100% of the original content of liothyronine sodium when stored under standard testing conditions at 25° C.±2° C. and 60%±5% relative humidity for a period of nine months. Even when tablets were stored under accelerated conditions (40° C.±2° C. and 75%±5% relative humidity) for six months, tablets produced in Batch NB543-050 retained 100% of the original liothyronine sodium content. When tablets were stored under extreme conditions (60° C.±2° C.) for 15 days, tablets produced in Batch NB543-050 retained about 99.5% of the original liothyronine sodium content.

### Example 4

A study was conducted to assess the stability of embodiments of pharmaceutical oral solid dosage forms according to the present disclosure.

Tablets including about 37.5 μg liothyronine sodium and about 150 g levothyroxine sodium were prepared by compressing portions of several uniform mixtures made using the method generally described in Example 1. Each of the mixtures included liothyronine sodium, levothyroxine sodium, MCC, mannitol, hydroxypropyl methylcellulose, butylhydroxyanisole, talc, and magnesium stearate. The MCC was included as a component of Avicel® HFE 102 powder. Table 4 below provides the mass and weight/weight concentration of the ingredients in individual tablets comprising about 37.5 μg liothyronine sodium and about 150 μg levothyroxine sodium per tablet produced in Batch NB543-051.

TABLE 4
Composition of tablets in Batch NB543-051

| Ingredient | % w/w | mg/unit |
|---|---|---|
| Liothyronine sodium, USP | 0.0288 | 0.0375 |
| Levothyroxine sodium, USP | 0.115 | 0.15 |
| Avicel ® HFE 102 Powder | 84.57 | 109.938 |
| Mannitol, USP | 11.27 | 14.65 |
| Hydroxypropyl | 2.31 | 3.00 |
| methylcellulose, NF | | |
| Butylhydroxyanisole, NF | 0.00384 | 0.005 |
| Talc, NF | 0.77 | 1.00 |
| Magnesium Stearate, NF | 0.77 | 1.00 |
| FD&C Blue #1 Aluminum | — | — |
| Lake | | |
| D&C Yellow #10 Aluminum | 0.15 | 0.2 |
| Lake | | |
| FD&C Red #40 Aluminum | 0.015 | 0.02 |
| Lake | | |
| Total | 100.00 | 130 |

Solid dosage forms including different amounts of the active agents (levothyroxine sodium and liothyronine sodium) may be formed in a dose similar manner by adjusting the amount of the active ingredient, along with the amount of inactive ingredients to keep the mass of the dosage forms the same.

For purposes of conducting the stability study, the tablets of Batch NB543-051 were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed with a closure. As an alternative, air may be displaced within a container by disposing an inert pill packing material, for example, rayon or cotton, in the container after tablet loading and before induction sealing.

Four studies were conducted to assess the stability of the levothyroxine sodium and liothyronine sodium in the tablets produced in Batch NB543-051. The stability studies involved the following testing periods and conditions:
Storing under refrigerated conditions (2° C. to 8° C.) for up to nine months.
Storing under standard testing conditions (25° C.±2° C. and 60%±5% relative humidity) for up to nine months.
Storing under accelerated testing conditions (40° C.±2° C. and 75%±5% relative humidity) for up to six months.
Storing under extreme testing conditions (60° C.±2° C.) for 7 or 15 days.

A refrigerated testing conditions stability study of the tablets of Batch NB543-051 contained in the container closure system was conducted over a period of nine months to determine the rate of physical or chemical degradation of the levothyroxine sodium and liothyronine sodium included in tablets when stored under the refrigerated environmental conditions.

Refrigerated condition stability samples were placed upright in an environmental chamber and maintained in an upright position during the stability testing. As discussed, each tablet was disposed in the sealed container containing either 10 to 20 or 90 to 100 tablets from Batch NB543-051, and each tablet included about 37.5 μg liothyronine sodium and about 150 μg levothyroxine sodium per tablet. The sealed containers were maintained at 2° C. to 8° C., uninterrupted, (except for the addition or withdrawal of test samples) for a period of one month, three months, six months, or nine months. Tablets were removed from the storage conditions at the specified time points and tested for levothyroxine and liothyronine content using high pressure liquid chromatography.

Considering levothyroxine sodium content, shelf life of tablets of Batch NB543-051 was estimated to be at least nine months when stored under refrigerated testing conditions. Table 5 provides the nine-month stability assay results for tablets of Batch NB543-051 stored under refrigerated testing conditions. The stability assay showed that greater than 95 percent of the original levothyroxine sodium content was retained in tablets stored under refrigerated testing conditions for nine months.

Considering liothyronine sodium content, shelf life of tablets of Batch NB543-051 was estimated to be at least nine months when stored under refrigerated testing conditions. As shown in Table 5, the stability assay showed that greater than 95 percent of the original liothyronine sodium content was retained in tablets stored under refrigerated testing conditions for nine months.

A standard testing conditions stability study of the tablets of Batch NB543-051 contained in the container closure system was conducted over a period of nine months to determine the rate of physical or chemical degradation of the levothyroxine sodium and liothyronine sodium included in tablets when stored under the standard environmental conditions.

Container closure systems prepared as described above containing tablets including about 37.5 μg liothyronine sodium and about 150 μg levothyroxine sodium per tablet were placed upright in a calibrated environmental chamber and maintained in an upright position during the stability study. The sealed containers including either 10 to 20 or 90 to 100 tablets were maintained at 25° C.±2° C. and 60%±5% relative humidity, uninterrupted (except for the addition or withdrawal of test samples), for a period of one month, two months, three months, six months, or nine months. Tablets were removed from the storage conditions at the specified time points and tested for levothyroxine sodium and liothyronine sodium content using high pressure liquid chromatography.

Considering levothyroxine sodium content, shelf life of tablets of Batch NB543-051 was estimated to be at least nine months when stored under standard testing conditions. Table 5 provides the nine-month stability assay results for tablets of Batch NB543-051. The stability assay showed that greater than 95 percent of the original levothyroxine sodium content was retained in tablets stored under standard testing conditions for nine months.

Considering liothyronine sodium content, shelf life of tablets of Batch NB543-051 was estimated to be at least nine months when stored under standard testing conditions. As shown in Table 5, the stability assay showed that greater than 95 percent of the original liothyronine sodium content was retained in tablets stored under standard testing conditions for nine months.

Accelerated condition stability samples were placed upright in an environmental chamber and maintained in an upright position during the stability testing. As discussed, each tablet was disposed in the sealed container containing either 10 to 20 or 90 to 100 tablets from Batch NB543-051, and each tablet included about 37.5 μg liothyronine sodium and about 150 μg levothyroxine sodium per tablet. The sealed containers were maintained at 40° C.±2° C. and 75%±5% relative humidity, uninterrupted, (except for the addition or withdrawal of test samples) for a period of one month, two months, three months, or six months. Tablets were removed from the storage conditions at the specified time points and tested for levothyroxine sodium and liothyronine sodium contents using high pressure liquid chromatography.

Considering levothyroxine sodium content, shelf life of tablets of Batch NB543-051 was estimated to be at least six months when stored under accelerated testing conditions. Table 5 provides the six-month stability assay results for tablets of Batch NB543-051 stored under accelerated testing conditions. The stability assay showed that greater than 95 percent of the original levothyroxine sodium content was retained in tablets stored under accelerated testing conditions for six months.

Considering liothyronine sodium content, shelf life of tablets of Batch NB543-051 was estimated to be at least six months when stored under accelerated testing conditions. As shown in Table 5, the stability assay showed that greater than 95 percent of the original liothyronine sodium content was retained in tablets stored under accelerated testing conditions for six months.

An extreme testing conditions stability study was conducted in which tablets of Batch NB543-051 were stored under extreme testing conditions for a period of 7 or 15 days to determine the rate of physical or chemical degradation of the original content of about 37.5 μg liothyronine sodium and about 150 μg levothyroxine sodium in the tablets. All extreme testing conditions stability samples (each sample comprising a container closure system containing either 10 to 20 or 90 to 100 tablets of Batch NB543-051) were placed upright in an environmental chamber and maintained in an upright position during the stability study. All of the containers were maintained at 60° C.±2° C., uninterrupted (except for the addition or withdrawal of test samples), for a period of 7 or 15 days. Tablets were removed from the storage conditions at the specified time points and tested for levothyroxine sodium and liothyronine sodium contents using high pressure liquid chromatography.

Considering levothyroxine sodium content, shelf life of tablets of Batch NB543-051 was estimated to be at least fifteen days when stored under extreme testing conditions. Table 5 provides the fifteen-day stability assay results for tablets of Batch NB543-051. The stability assay showed that greater than 95 percent of the original levothyroxine sodium content was retained in tablets stored under extreme testing conditions for fifteen days.

Considering liothyronine sodium content, shelf life of tablets of Batch NB543-051 was estimated to be at least fifteen days when stored under extreme testing conditions. As shown in Table 5, the stability assay showed that greater than 95 percent of the original liothyronine sodium content was retained in tablets stored under extreme testing conditions for fifteen days.

Table 5 reports stability data for the pharmaceutical oral solid dosage form samples (Batch NB543-051) tested under refrigerated, standard, accelerated, and extreme conditions. The assay percentages listed in Table 4 are based on expected (“label claim”) contents of about 37.5 μg liothyronine sodium and about 150 μg levothyroxine sodium per tablet of Batch NB543-051.

TABLE 5
Stability assay results for tablets of Batch NB543-051

Attribute
Assay % Label Claim

| Stability | Levothyroxine | Liothyronine |
|---|---|---|
| Condition | Sodium | Sodium |
| T0 | 97.6 | 99.4 |
| 7 days/60 °C | 95.7 | 97.3 |
| 15 days/60 °C | 94.4 | 96.4 |
| 1 M, 2 °C-8°C | 97.3 | 99.0 |
| 3 M, 2 °C-8°C | 98.4 | 99.7 |
| 6 M, 2 °C-8 °C | 97.5 | 99.5 |
| 9 M, 2 °C-8 °C | 98.2 | 98.9 |
| 1 M, 25 °C/60% RH | 97.1 | 98.8 |
| 2 M, 25 °C/60% RH | 98.7 | 98.1 |
| 3 M, 25 °C/60% RH | 98.5 | 100.1 |
| 6 M, 25 °C/60% RH | 97.8 | 99.7 |
| 9 M, 25 °C/60% RH | 97.7 | 98.5 |
| 1 M, 40 °C/75% RH | 96.4 | 98.1 |
| 2 M, 40 °C/75% RH | 98.0 | 97.8 |
| 3 M, 40 °C/75% RH | 97.0 | 98.8 |
| 6 M, 40 °C/75% RH | 97.5 | 99.9 |

With reference to the results shown in Table 5, the present inventors surprisingly observed that pharmaceutical oral solid dosage forms produced in Batch NB543-051 retained 100% of the original content of levothyroxine sodium when stored under refrigerated testing conditions at 2° C. to 8° C. for a period of nine months, and also retained about 100% of the original content of levothyroxine sodium when stored under standard testing conditions at 25° C.±2° C. and 60%±5% relative humidity for a period of nine months. Even when tablets were stored under accelerated conditions (40° C.±2° C. and 75%±5% relative humidity) for six months, tablets produced in Batch NB543-051 retained about 100% of the original levothyroxine sodium content (a decrease in the levothyroxine content of the tablets of about two percent). When tablets were stored under extreme conditions (60° C.±2° C.) for 15 days, tablets produced in Batch NB543-051 retained about 97% of the original levothyroxine sodium content.

With reference to the results shown in Table 5, the present inventors also surprisingly observed that pharmaceutical oral solid dosage forms produced in Batch NB543-051 retained 99% of the original content of liothyronine sodium when stored under refrigerated testing conditions at 2° C. to 8° C. for a period of nine months and also retained 99% of the original content of liothyronine sodium when stored under standard testing conditions at 25° C.±2° C. and 60%±5% relative humidity for a period of nine months. Even when tablets were stored under accelerated conditions (40° C.±2° C. and 75%±5% relative humidity) for six months, tablets produced in Batch NB543-051 retained 100% of the original liothyronine content. When tablets were stored under extreme conditions (60° C.±2° C.) for 15 days, tablets produced in Batch NB543-051 retained about 97% of the original liothyronine content.

### Example 5

A study was conducted to assess the stability of embodiments of pharmaceutical oral solid dosage forms according to the present disclosure.

Tablets including about 62.5 μg liothyronine sodium and about 250 μg levothyroxine sodium were prepared by compressing portions of several uniform mixtures made using the method generally described in Example 1. Each of the mixtures included liothyronine sodium, levothyroxine sodium, MCC, mannitol, hydroxypropyl methylcellulose, butylhydroxyanisole, talc, and magnesium stearate. The MCC was included as a component of the Avicel® HFE 102 powder in the mixture. Table 6 below provides the mass and weight/weight concentration of the ingredients in individual tablets comprising about 62.5 μg liothyronine sodium and about 250 μg levothyroxine sodium per tablet produced in Batch NB543-098.

TABLE 6
Composition of tablets in Batch NB543-098

| Ingredient | % w/w | mg/unit |
|---|---|---|
| Liothyronine sodium, USP | 0.048 | 0.0625 |
| Levothyroxine sodium, USP | 0.192 | 0.250 |
| Avicel ® HFE 102 Powder | 82.33 | 107.032 |
| Mannitol, USP | 11.27 | 14.65 |
| Hydroxypropyl | 4.62 | 6.00 |
| methylcellulose, NF | | |
| Butylhydroxyanisole, NF | 0.00384 | 0.005 |
| Talc, NF | 0.77 | 1.00 |
| Magnesium Stearate, NF | 0.77 | 1.00 |
| FD&C Blue #1 Aluminum | — | — |
| Lake | | |
| D&C Yellow #10 Aluminum | — | — |
| Lake | | |
| FD&C Red #40 Aluminum | — | — |
| Lake | | |
| Total | 100.00 | 130 |

Solid dosage forms including different amounts of the active agents (levothyroxine sodium and liothyronine sodium) may be formed in a dose similar manner by adjusting the amount of the active ingredient, along with the amount of inactive ingredients to keep the mass of the dosage forms the same.

For purposes of conducting the stability study, the tablets of Batch NB543-098 were disposed in container closure systems including a container comprising an internal volume of either about 30 cm³ (including 10 to 20 tablets) or about 75 cm³ (including 90 to 100 tablets) and a sealing closure. 0, 1, or 2 silica gel desiccant canisters (including, respectively, 0, 2, or 4 g of silica gel) and 0, 1, or 2 PHARMAKEEP® oxygen absorber canisters (including, respectively, 0, 2, or 4 g of oxygen absorber) were also included in each container. Once filled, each container was induction sealed with a closure. As an alternative, air may be displaced within a container by disposing an inert pill packing material, for example, rayon or cotton, in the container after tablet loading and before induction sealing.

One study was conducted to assess the stability of the levothyroxine sodium and liothyronine sodium in the tablets produced in Batch NB543-098. The stability studies involved the following testing periods and conditions:
Storing under accelerated testing conditions (40° C.±2° C. and 75%±5% relative humidity) for up to two months.

An accelerated testing conditions stability study of the tablets of Batch NB543-098 contained in the container closure system was conducted over a period of two months to determine the rate of physical or chemical degradation of the levothyroxine sodium and liothyronine sodium included in tablets when stored under the accelerated environmental conditions.

Container closure systems prepared as described above containing tablets including about 62.5 μg liothyronine sodium and about 250 μg levothyroxine sodium per tablet were placed upright in a calibrated environmental chamber and maintained in an upright position during the stability study. The sealed containers including either 10 to 20 or 90 to 100 tablets were maintained at 40° C.±2° C. and 75%±5% relative humidity, uninterrupted (except for the addition or withdrawal of test samples), for a period of one month or two months. Tablets were removed from the storage conditions at the specified time points and tested for levothyroxine sodium and liothyronine sodium contents using high pressure liquid chromatography.

Considering levothyroxine sodium content, shelf life of tablets of Batch NB543-098 was estimated to be at least two months when stored under accelerated testing conditions. Table 7 provides the two month stability assay results for tablets of Batch NB543-098 stored under accelerated testing conditions. The stability assay showed that greater than 95 percent of the original levothyroxine sodium content was retained in tablets stored under accelerated testing conditions for two months.

Considering liothyronine sodium content, shelf life of tablets of Batch NB543-098 was estimated to be at least two months when stored under accelerated testing conditions. As shown in Table 7, the stability assay showed that greater than 95 percent of the original liothyronine sodium content was retained in tablets stored under accelerated testing conditions for two months.

Table 7 reports stability data for the pharmaceutical oral solid dosage form samples (Batch NB543-098) tested under accelerated conditions. The assay percentages listed in Table 7 are based on expected (“label claim”) contents of about 62.5 μg liothyronine sodium and about 250 μg levothyroxine sodium per tablet of Batch NB543-098.

TABLE 7
Stability assay results for tablets of Batch NB543-098

| | | | | | % Assay | |
|---|---|---|---|---|---|---|
| | | Desiccant- | Desiccant- | Nitrogen | Levothyroxine | Liothyronine |
| Time Point | Bottle | Sorbit | Pharmakeep | (N₂) | Sodium | Sodium |
| Time Zero | NA | NA | NA | NA | 101.3 | 98.2 |
| 1 M, 40 °C/ | 100 | 2 | 2 | Yes | 100.2 | 98.2 |
| 75% RH | tablets | 2 | 1 | Yes | 100.5 | 97.7 |
| | | 2 | 0 | Yes | 99.6 | 97.7 |
| | | 2 | 2 | No | 100.6 | 97.6 |
| | | 2 | 1 | No | 99.8 | 97.9 |
| | | 2 | 0 | No | 99.4 | 97.8 |
| | | 0 | 0 | No | 99.9 | 97.6 |
| 2 M, 40 °C/ | 100 | 2 | 2 | Yes | 99.9 | 97.7 |
| 75% RH | tablets | 2 | 1 | Yes | 99.9 | 97.5 |
| | | 2 | 0 | Yes | 98.4 | 96.5 |
| | | 2 | 2 | No | 99.1 | 97.3 |
| | | 2 | 1 | No | 99.8 | 97.4 |
| | | 2 | 0 | No | 98.8 | 96.5 |
| | | 0 | 0 | No | 97.3 | 94.7 |
| 1 M, 40 °C/ | 14 | 1 | 1 | Yes | 100.6 | 97.6 |
| 75% RH | tablets | 1 | 0 | Yes | 99.1 | 97.6 |
| | | 1 | 1 | No | 99.9 | 97.7 |
| | | 1 | 0 | No | 98.2 | 97.2 |
| | | 0 | 0 | No | 98.2 | 95.3 |
| 2 M, 40 °C/ | 14 | 1 | 1 | Yes | 99.7 | 97.0 |
| 75% RH | tablets | 1 | 0 | Yes | 98.0 | 96.7 |
| | | 1 | 1 | No | 99.8 | 97.4 |
| | | 1 | 0 | No | 97.5 | 97.0 |
| | | 0 | 0 | No | 97.5 | 95.2 |

With reference to the results shown in Table 7, the present inventors surprisingly observed that pharmaceutical oral solid dosage forms produced in Batch NB543-098 retained about 96% to about 99% of the original content of levothyroxine sodium when stored under accelerated conditions (40° C.±2° C. and 75%±5% relative humidity) for two months.

With reference to the results shown in Table 7, the present inventors also surprisingly observed that pharmaceutical oral solid dosage forms produced in Batch NB543-098 retained about 96% to about 99% of the original content of liothyronine sodium when stored under accelerated conditions (40° C.±2° C. and 75%±5% relative humidity) for two months.

### Example 6

This example describes a non-limiting embodiment of a dry layering/mixing and tableting process that may be used to make embodiments of pharmaceutical oral solid dosage forms according to the present disclosure.

Thirty-three percent of a total required amount of AVICEL® PH-102 MCC powder was screened through a #30 mesh screen, and the screened material was then mixed in a high shear mixer granulator (HSMG) (Glatt VG PRO) for thirty seconds at 250 rpm per minute with amperage at 3-4 amperes. The screened and mixed portion of the AVICEL® PH-102 powder formed a first layer in the HSMG.

Next, a screened blend was prepared by dry screening through a #30 mesh screen into a vessel the remaining 75% of the total needed amount of AVICEL® PH-102 powder, levothyroxine sodium (previously screened through a #60 mesh screen), liothyronine sodium (previously screened through a #200 mesh screen), butylhydroxyanisole (previously screened through a #200 mesh screen), mannitol (previously screened through a #60 mesh screen), coloring agents (previously screened through a #60 mesh screen), and talc (previously screened through a #30 mesh screen). The screened levothyroxine sodium and liothyronine sodium were sandwiched in the HSMG between the layers of screened AVICEL® PH-102 powder. Screened butylhydroxyanisole was then added, followed by screened mannitol, screen coloring agents, and screened talc. This screened blend was positioned in the HSMG to form a second layer adjacent to the first (AVICEL® PH-102 powder) layer.

The assembly of two layers disposed in the HSMG constituted a dry layered blend. The HSMG was activated to uniformly mix the dry layered blend for five minutes using an HSMG at 250 rpm per minute with amperage at 3-4 amperes. The HSMG was operated under low purge air pressure of 0.02 psi. The mixed dry layered blend was removed from the HSMG and screened through a #30 mesh screen and returned to the HSMG for further uniform mixing for 4 minutes using an HSMG at 250 rpm per minute with amperage at 3-4 amperes.

Magnesium stearate was screened through a #30 mesh screen and added to the doubly mixed and screened dry layered blend, and the materials were uniformly mixed for 60 seconds using an HSMG at 460 rpm per minute with amperage at 3-4 amperes to form a final oral dosage mixture.

Portions of the final oral dosage mixture were removed from the HSMG using a cradle discharge method. The cradle discharge method removed multiple small quantities of the final oral dosage mixture from the HSMG over short time periods to prevent segregation of the ingredients. Finally, measured portions of the dry oral dosage mixture were compressed into 130 mg tablets using a Piccola/Globe pharma tablet press. These were compressed to a hardness of 4-8 Kp.

The tablets were then packaged in either 30 cm³ or 75 cm³ high density polyethylene bottles.

About 10 to about 20 of the tablets and 0, 1, or 2 silica gel desiccant canisters (commercially available from, e.g., Clariant International, Colton, CA) (each canister including about 2 g of desiccant) were disposed in the 30 cm³ HDPE containers (shown in FIGS. 1A, 1B, and 1C). In certain of the 30 cm³ containers including about 10 to 20 tablets, 0, 1, or 2 PHARMAKEEP® oxygen absorber canisters (commercially available from Clariant International) (each canister including about 2 g of oxygen absorber) also were disposed. The 30 cm³ containers were sealed with a sealing closure (depicted in FIGS. 2A, 2B, and 2C), providing a packaged pharmaceutical oral solid dosage form product.

About 90 to about 110 the tablets and 0, 1, or 2 of the silica gel desiccant canisters were disposed in the 75 cm³ containers (shown in FIGS. 3A, 3B, and 3C). In certain of the 75 cm³ containers including about 90 to 110 tablets, 0, 1, or 2 of the PHARMAKEEP® oxygen absorber canisters also were disposed. The 75 cm³ containers were sealed with a sealing closure (shown in FIGS. 4A and 4B), providing a packaged pharmaceutical oral solid dosage form product.

### Example 7

Tablets individually including about 2.25 μg to about 62.5 μg liothyronine sodium and about 9.5 μg to about 250 μg levothyroxine sodium were prepared by compressing portions of several uniform mixtures made using the method generally described in Example 6. Each of the tablets included liothyronine sodium, levothyroxine sodium, microcrystalline cellulose (MCC), mannitol, butylhydroxyanisole, talc, a coloring agent, and magnesium stearate. Table 8 below provides the mass of the ingredients in individual tablets comprising about 2.25 μg to about 62.5 μg liothyronine sodium and

1. A ready-to-use pharmaceutical oral solid dosage form comprising:
11 to 15 weight percent non-blended porcine thyroid powder;
57 to 70 weight percent microcrystalline cellulose; and
at least one excipient selected from a lubricant, a glidant, a diluent, and a disintegrant.

2. The ready-to-use pharmaceutical oral solid dosage form of claim 1, wherein the non-blended porcine thyroid powder comprises 7 μg to 230 μg of levothyroxine.

3. The ready-to-use pharmaceutical oral solid dosage claim 1, wherein the non-blended porcine thyroid powder comprises 1.6 μg to 55 μg of liothyronine.

4.-17. (canceled)

18. The ready-to-use pharmaceutical oral solid dosage claim 1, wherein the pharmaceutical solid dosage form comprises mannitol.

19. (canceled)

20. The ready-to-use pharmaceutical oral solid dosage claim 1, wherein the pharmaceutical solid dosage form comprises at least one lubricant selected from calcium stearate and magnesium stearate.

21. The ready-to-use pharmaceutical oral solid dosage claim 1, wherein the pharmaceutical solid dosage form comprises, by weight, 1.5% to 2.5% colloidal silicon dioxide.

22. The ready-to-use pharmaceutical oral solid dosage claim 1, wherein the pharmaceutical solid dosage form comprises, by weight, 12% to 14% non-blended porcine thyroid powder.

23. The ready-to-use pharmaceutical oral solid dosage form of claim 1, wherein the pharmaceutical solid dosage form comprises, by weight, 62% to 66% microcrystalline cellulose.

24. A ready-to-use pharmaceutical oral solid dosage form comprising:
11 to 15 weight percent non-blended porcine thyroid powder;
57 to 70 weight percent microcrystalline cellulose;
18 to 22 weight percent mannitol;
1.5 to 2.5 weight percent colloidal silicon dioxide; and
calcium stearate.

25. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 3 months at a temperature of about 23° C. to about 27° C. and a relative humidity ranging from about 55% to about 65%.

26. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 6 months at a temperature of about 23° C. to about 27° C. and a relative humidity ranging from about 55% to about 65%.

27. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 9 months at a temperature of about 23° C. to about 27° C. and a relative humidity ranging from about 55% to about 65%.

28. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 12 months at a temperature of about 23° C. to about 27° C. and a relative humidity ranging from about 55% to about 65%.

29. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 18 months at a temperature of about 23° C. to about 27° C. and a relative humidity ranging from about 55% to about 65%.

30. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 3 months at a temperature ranging from about 38° C. to about 42° C. and a relative humidity ranging from about 70% to about 80%.

31. The ready-to-use pharmaceutical oral solid dosage claim 1, provided that a levothyroxine content in the pharmaceutical solid dosage form is at least 90% of an initial levothyroxine content in the pharmaceutical solid dosage form after the pharmaceutical solid dosage form has been stored for 6 months at a temperature ranging from about 38° C. to about 42° C. and a relative humidity ranging from about 70% to about 80%.

32. The ready-to-use pharmaceutical oral solid dosage claim 1, provided that a liothyronine content in the pharmaceutical solid dosage form is at least 90% of an initial liothyronine content in the pharmaceutical solid dosage form after the pharmaceutical solid dosage form has been stored for 3 months at a temperature of about 23° C. to about 27° C. and a relative humidity ranging from about 55% to about 65%.

33. The ready-to-use pharmaceutical oral solid dosage claim 1, provided that a liothyronine content in the pharmaceutical solid dosage form is at least 90% of an initial liothyronine content in the pharmaceutical solid dosage form after the pharmaceutical solid dosage form has been stored for 6 months at a temperature of about 23° C. to about 27° C. and a relative humidity ranging from about 55% to about 65%.

34. The ready-to-use pharmaceutical oral solid dosage claim 1, provided that the liothyronine content in the pharmaceutical solid dosage form is at least 90% of an initial liothyronine content in the pharmaceutical solid dosage form after the pharmaceutical solid dosage form has been stored for 9 months at a temperature of about 23° C. to about 27° C. and a relative humidity ranging from about 55% to about 65%.

35. The ready-to-use pharmaceutical oral solid dosage claim 1, provided that the liothyronine content in the pharmaceutical solid dosage form is at least 90% of an initial liothyronine content in the pharmaceutical solid dosage form after the pharmaceutical solid dosage form has been stored for 12 months at a temperature of about 23° C. to about 27° C. and a relative humidity ranging from about 55% to about 65%.

36. The ready-to-use pharmaceutical oral solid dosage claim 1, provided that the liothyronine content in the pharmaceutical solid dosage form is at least 90% of an initial liothyronine content in the pharmaceutical solid dosage form after the pharmaceutical solid dosage form has been stored for 18 months at a temperature of about 23° C. to about 27° C. and a relative humidity ranging from about 55% to about 65%.

37. The ready-to-use pharmaceutical oral solid dosage claim 1, provided that a liothyronine content in the pharmaceutical solid dosage form is at least 90% of an initial liothyronine content in the pharmaceutical solid dosage form after the pharmaceutical solid dosage form has been stored for 3 months at a temperature ranging from about 38° C. to about 42° C. and a relative humidity ranging from about 70% to about 80%.

38. The ready-to-use pharmaceutical oral solid dosage claim 1, provided that the liothyronine content in the pharmaceutical solid dosage form is at least 90% of an initial liothyronine content in the pharmaceutical solid dosage form after the pharmaceutical solid dosage form has been stored for 6 months at a temperature ranging from about 38° C. to about 42° C. and a relative humidity ranging from about 70% to about 80%.

39.-42. (canceled)

43. The ready-to-use pharmaceutical oral solid dosage claim 1, provided that when a plurality of the pharmaceutical oral solid dosage forms and at least about 2 grams of desiccant are placed in a container sealed with a closure, each oral solid dosage form retains at least 90% of an initial levothyroxine content in the oral solid dosage form after the oral solid dosage form has been stored for 18 months at a temperature ranging from about 23° C. to about 27° C. and a relative humidity ranging from about 55% to about 65%.

44. (canceled)

45. The ready-to-use pharmaceutical oral solid dosage claim 1, provided that when a plurality of the pharmaceutical oral solid dosage forms and at least about 2 grams of desiccant are placed in a container sealed with a closure, each oral solid dosage form retains at least 90% of an initial levothyroxine content in the oral solid dosage form after the oral solid dosage form has been stored for 6 months at a temperature ranging from 38° C. to about 42° C. and a relative humidity ranging from about 70% to about 80%.

46.-49. (canceled)

50. The ready-to-use pharmaceutical oral solid dosage claim 1, provided that when a plurality of the pharmaceutical oral solid dosage forms and at least about 2 grams of desiccant are placed in a container sealed with a closure, each oral solid dosage form retains at least 90% of an initial liothyronine content in the oral solid dosage form after the oral solid dosage form has been stored for 18 months at a temperature ranging from about 23° C. to about 27° C. and a relative humidity ranging from about 55% to about 65%.

51. (canceled)

52. The ready-to-use pharmaceutical oral solid dosage claim 1, provided that when a plurality of the pharmaceutical oral solid dosage forms and at least about 2 grams of desiccant are placed in a container sealed with a closure, each oral solid dosage form retains at least 90% of an initial liothyronine content in the oral solid dosage form after the oral solid dosage form has been stored for 6 months at a temperature ranging from 38° C. to about 42° C. and a relative humidity ranging from about 70% to about 80%.

53.-100. (canceled)

101. A ready-to-use pharmaceutical oral solid dosage form comprising:
12 to 14 weight percent non-blended porcine thyroid powder;
62 to 66 weight percent microcrystalline cellulose;
18 to 22 weight percent mannitol;
1.5 to 2.5 weight percent colloidal silicon dioxide; and
calcium stearate.

102. The ready-to-use pharmaceutical oral solid dosage form of claim 101, wherein the non-blended porcine thyroid powder comprises 7 μg to 230 μg of levothyroxine.

103. The ready-to-use pharmaceutical oral solid dosage form of claim 101, wherein the non-blended porcine thyroid powder comprises 1.6 μg to 55 μg of liothyronine.
